(19) United States
(12) Patent Application Publication
(10) Pub. No.: US 2008/0312425 A1
(43) Pub. Date: Dec. 18, 2008

(54) Ion Exchange Chromatography and Purification of Antibodies

(75) Inventors: Julian Bonnerjea, Bucks (GB); Robert P. Brake, Dover, NH (US); Mark R. Davis, Portsmouth, NH (US); Keith Kellerman, Rochester, NH (US); Anna Preneta, Surrey (GB)

Correspondence Address: Warren M. Check, Jr.;Wenderoth, Lind & Ponack, L.L.P., 2033 K Street, N.W., Suite 800, Washington, DC, 20006, US

(73) Assignee: Lonza Biologics plc., Slough (GB)

(21) Appl. No.: 10/595,671

(22) PCT Filed: Aug. 30, 2005
(86) PCT No.: PCT/EP05/09343

§ 371 Date: May 3, 2006

Related U.S. Application Data
(60) Provisional application No. 60/605,171, filed on Aug. 30, 2004.
(60) Provisional application No. 60/608,104, filed on Sep. 9, 2004.

(51) Int. Cl. C07K 1/00

(52) U.S. Cl. 530/413

(57) ABSTRACT

A novel method for purifying antibody and other product protein concomitant with removing aggregates made up from single product protein species is devised.

The present invention relates to the field of protein and in particular antibody purification in biotechnological production. It is an object of the present invention to describe a novel process for purification of such protein or antibody.

Protein A chromatography is widely used in industrial manufacturing of antibodies since allowing for almost complete purification of antibodies, that is usually IgG, in a single step from cell culture supernatants. Protein A affinity columns inevitably are subject to some degree of leakage of ligand from the column upon repeated runs. Partly, this may be due to proteolytic clipping of protein A from the column; in industrial manufacture of antibody for pharmaceutical applications, no protease inhibitor cocktails may be added for regulatory reasons. Unfortunately, this protein A or protein A fragment contaminants retain their affinity for IgG and are difficult to remove from the purified antibody due to ongoing complex formation. Removal of such heterogenous dimeric complexes of two different macromolecules from purified antibody is mandatory since protein A which is a bacterial protein will elicit an unwanted immune response; model complexes formed by adding protein A to monomeric IgG have been reported to activate Fc-bearing leukocytes and the complement system to generate oxidant and anaphylatoxins activity in vitro (Balint et al., Cancer Res. 44, 734, 1984). Balint et al. (supra.) and others (Das et al., 1985, Analyt. Biochem. 145, 27-36) demonstrated that such IgG-Protein complexes can be separated from uncomplexed IgG by gel filtration. Low through-put and loss in antibody yield are the disadvantages of this method.

The more recent commercialization of recombinant Protein A species attached to the column matrix via a single thioester bond allows for higher capacity protein A columns as set forth in U.S. Pat. No. 6,399,750. Concomitantly, the leakage rate of such recombinant Protein A matrices is often drastically increased in contrast to traditional, multi-point attached natural Protein A matrices obtained by CNBr coupling.

U.S. Pat. No. 4,983,722 teaches selective separation of contaminating protein A from a protein-A purified antibody preparation by absorbing the mixture to an anion exchanger material and to separate both components by sequentially eluting the antibodies and protein A under conditions of increasing ionic strength. This resolution method is highly dependent on the pI of the antibody which is specific and highly variable for a given antibody. Further, throughput is limited by the slope of the salt gradient required for obtaining good separation.

Apart from removing protein A complexes, a further purification problem that relates to antibodies but beyond that to any other type of biopharmaceutical protein, is the formation of homogenous dimers and higher order aggregates. In contrast to complex formation with protein A, which is mainly affinity based and does even occur with native protein, homogenous chemical mass law driven-aggregates of antibody or similar protein start to form after spontaneous or salt or pH induced denaturing of at least parts of the protein, exposing hydrophobic patches on the solvent accessible surface. Hence non-specific aggregation, in contrast to affinity based complex formation, is mainly driven by solvent exclusion effects and resemble crystal growth behaviour in this regard. The initially still soluble aggregates may increase with time and give rise to precipitation of protein from solution. Upon pharmaceutical dosing, low percentages of contaminating aggregates further elicit unwanted immune responses. Removing aggregates reliably was done so far almost exclusively by size exclusion chromatography (SEC); however, SEC is a bottleneck in purification requiring huge processing times, expensive materials and allow of low capacity loading only as compared to other chromatography techniques.

It is one object of the present invention to devise another method for separating protein A or protein A fragments from antibody, preferably an IgG, and/or for separating antibody aggregates or homogenous aggregates of other product protein to be purified which method avoids the disadvantages of the prior art. This objects are solved by the methods of the present invention.

According to the present invention, a method of purifying an antibody is devised which method comprises the steps of:

Firstly, purifying an antibody by means of protein A affinity chromatography wherein the protein A is a native protein A or a functional derivative thereof and,
Secondly, loading the thus purified antibody comprising antibody aggregate and protein A or protein A derivative onto an ion exchange material under conditions which allow of binding of the contaminating protein A or its functional derivative to the ion exchanger material and which conditions further allow of resolution in the flow-through by means of fractionation of the flow-through of antibody aggregates from antibody monomer which monomer is not complexed with protein A or protein A derivative and thirdly, fourthly fractionating the flow-through and harvesting from the flow-through of the ion exchanger at least one antibody monomer fraction having both reduced contents of protein A or protein A derivative and further reduced contents of antibody aggregate as compared to the composition of antibody as loaded onto the ion exchange material before.

Preferably, the method of the present invention reduces the aggregate contents of the antibody monomer thus purified to below 1.0%, more preferably to below 0.5% of all antibody finally collected in the flow-through from said or first ion exchange step. Hence the monomericity of the antibody as obtained after the ion exchange step according to the method of the present invention is at least 99%, more preferably is at least 99.5%, as may be determined by analytical size exclusion chromatography well known to the skilled person.

Further preferred is collecting in said harvest fraction of the flow-through at least 70%, more preferably collecting at least 80%, most preferably collecting at least 90% of the total amount of antibody loaded onto the ion exchange material in the flow-through of the ion exchanger whilst any contaminant protein A or protein A derivative is bound to the ion exchange material.

An aggregate according to the present invention is understood as the non-covalent association of identical protein entities, preferably an association with an binding equilibrium constant of at least 10 exp-7 M or below (below in sense of tighter binding) which protein may be made up from single protein chains or from covalently bonded, e.g. bonded by means of disulfide bonds, homologous or heterologous multiple polypeptides. The aggregates to which the invention is referring to are soluble in aqueous solution just as are the monomers they are derived from. For instance, a ‘monomer’ of an IgG antibody according to the present invention relates to the standard tetrameric antibody comprising two identical, glycosylated Heavy and Light chains respectively. An e.g. dimeric aggregate is then the non-specific association of two IgG molecules. Aggregate formation is tightly linked to denaturating influences on the native protein fold and quaternary structure of proteins; aggregation may be e.g. elicited by thermal and pH-induced denaturation of the protein fold. Aggregation rate is hence highly specific for a given protein, depending on the energetic stability of the individual protein fold against a specific challenge (Chiti et al., 2004, Rationalization of the effects of mutations on protein aggregation rates, Nature 424: 805-808).

Protein A is a cell surface protein found in Staphylococcus aureus. It has the property of binding the Fc region of a mammalian antibody, in particular of IgG class antibodies. Within a given class of antibodies, the affinity slightly varies with regard to species origin and antibody subclass or allotype (reviewed in Surolia, A. et al., 1982, Protein A: Nature's universal antibody', TIBS 7, 74-76; Langone et al., 1982, Protein A of staphylococcus aureus and related immunoglobulin receptors, Advances in Immunology 32:157-252). Protein A can be isolated directly from cultures of S. aureus that are secreting protein A or is more conveniently recombinantly expressed in E. coli (Lofdahl et al., 1983, Proc. Natl. Acad. Sci. USA 80:697-701). Its use for purification of antibodies, in particular monoclonal IgG, is amply described in the prior art (e.g. Langone et al., supra; Hjelm et al, 1972; FEBS Lett. 28: 73-76). For use in protein A affinity chromatography, protein A is coupled to a solid matrix such as crosslinked, uncharged agarose (Sepharose, freed from the charged fraction comprised in natural unrefined agarose), trisacryl, crosslinked dextran or silica-based materials. Methods for such are commonly known in the art, e.g. coupling via primary amino functions of the protein to a CNBr-activated matrix. Protein A binds with high affinity and high specificity to the Fc portion of IgG, that is the C?2-Cy3 interface region of IgG as described in Langone et al., 1982, supra. In particular, it binds strongly to the human allotypes or subclasses IgG1, IgG2, IgG3 and the mouse allotypes or subclasses IgG2a, IgG2b, IgG3. Protein A also exhibits an affinity for the Fab region of immunoglobulins that are encoded by the V_{H} gene family, V_{H} III (Sasso et al., 1991, J. Immunol, 61: 3026-3031; Hilson et al., J Exp. Med., 178: 331-336 (1993)). The sequence of the gene coding for protein A revealed two functionally distinct regions (Uhlen et al., J. Biol. Chem., 259: 1695-1702 (1984); Lofdahl et al., Proc. Nutl. Acad. Sci. (USA), 80: 697-701 (1983)). The amino-terminal region contains five highly homologous IgG-binding domains (termed E, D, A, B and C), and the carboxy terminal region anchors the protein to the cell wall and membrane. All five IgG-binding domains of protein A bind to IgG via the Fc region, involving e.g. in human IgG-Fc residues 252-254, 433-435 and 311, as shown for the crystal structure in Deisenhofer et al. (1981, Biochemistry 20: 2361-2370) and in Sauer-Eriksson et al. (1995, Structure 3: 265-278) in case of the B-domain of protein A. The finding of two essentially contiguous main binding sites in the Fc portion has been confirmed in the NMR-solution study of Gouda et al., 1998, Biochemistry 37: 129-136. In principle, each of the IgG-binding domains A to E of protein A is sufficient for binding to the Fc-portion of an IgG. Further, certain human alleles of the VH3 domain family have been found to optionally mediate binding of human Ig by protein A (Ibrahim et al., 1993, J. Immunol. 151:3597-3603; V-region mediated binding of human Ig by protein A). In the context of the present application, in another, separate object of the present invention, everything that has been said applying to Fc-region binding of antibody to protein A applies likewise to the binding of antibodies via such VH3 family protein A-binding allele in case that the Fc-region of such antibody did not allow on itself for high-affinity protein A binding. It may be considered an equivalent embodiment of the principal, Fc-based method of the present invention; the latter is further described in the subsequent sections.

An IgG antibody according to the present invention is to be understood as an antibody of such allotype that it can be bound to protein A in a high-affinity mode. Further, apart from the Fc portions of the antibody that are relevant for binding to protein A, such antibody must not correspond to a naturally occurring antibody. In particular in its variable chain regions portions, it can be an engineered chimeric or CDR-grafted antibody as are routinely devised in the art. An IgG-antibody according to the present invention is to be understood as an IgG-type antibody, in short.

A functional derivative of protein A or protein A-fragment according to the present invention is characterized by a binding constant of at least K=10⁻⁸ M, preferably K=10⁻⁹ M for the Fc portion of mouse IgG2a or human IgG1. An interaction compliant with such value for the binding constant is termed ‘high affinity binding’ in the present context. Preferably, such functional derivative of protein A comprises at least part of a functional IgG binding domain of wild-type protein A which domain is selected from the natural domains E, D, A, B, C or engineered muteins thereof which have retained IgG binding functionality. An example of such is the functional 59 amino acid ‘Z’-fragment of domain B of protein A which domain may be used for antibody purification as set forth in U.S. Pat. No. 6,013,763. Preferably, however, an antibody binding fragment according to the present invention comprises at least two intact Fc binding domains as defined in this paragraph. An example of such are the recombinant protein A sequences disclosed e.g. in EP-282 308 and EP-284 368, both from Repligen Corporation.

Alone or in combination with a protein A or a functional protein A derivative as defined in the preceding sections, further preferred are protein A derivatives that are engineered to allow of single-point attachment. Single point attachment means that the protein moiety is attached via a single covalent bond to a chromatographic support material of the protein A affinity chromatography. Such single-point attachment by means of suitably reactive residues which further are ideally placed at an exposed amino acid position, namely in a loop, close to the N- or C-terminus or elsewhere on the outer circumference of the protein fold. Suitable reactive groups are e.g. sulfhydryl or amino functions. More preferably, such recombinant protein A or functional fragment thereof comprises a cysteine in its amino acid sequence. Most preferably, the cysteine is comprised in a segment that consists of the last 30 amino acids of the C-terminus of the amino acid sequence of the recombinant protein A or functional fragment thereof. In a further preferred embodiment of such type, the recombinant protein A or functional fragment thereof is attached by at least 50% via a thioether sulphur bond to the chromatographic support or matrix material of the protein A-affinity chromatography medium. An example of such an embodiment is described e.g. in U.S. Pat. No. 6,399,750 from Pharmacia and is commercially available under the brandnames of Streamline™ or MabSelect™ from Amersham-Biosciences, depending on the nature of the support matrix used. In the present context, thioether is to be understood narrowly as a —S-bonding scheme irrespective of chemical context, deviating in this regard from normal chemical language; it is possible, for instance, that said —S— ‘thioether’ bridge according to the present invention is part of a larger functional group such as e.g. a thioester or a mixed acetal, deviating in this regard in the context of the present application from the reactivity-based normal language of chemists. Preferably, the thioether bridge is a thioether bridge in its ordinary, narrow chemical meaning. Such bridging thioether group can be e.g. generated by reacting the sulfhydryl-group of a cysteine residue of the protein A with an epoxide group harbored on the activated chromatographic support material. With a terminal cysteine residue, such reaction can be carried out under conditions suitable as to allow only for coupling of an exposed, unique sulfhydryl group of a protein as to result in single-point attachment of such protein only.

In a particularly preferred embodiment, the protein A or functional protein A derivative according to the present invention is the recombinant protein A disclosed in U.S. Pat. No. 6,399,750 which comprises a juxtaterminal, engineered cysteine residue and is, preferably by at least 50%, more preferably by at least 70%, coupled to the chromatographic support material through the sulphur atom of said cysteine residue as the sole point of attachment. Further preferred, such coupling has been achieved by means of epoxide mediated activation, more preferably either by means of 1,4-bis-(2,3-epoxypropoxy) butane activation of e.g. an agarose matrix such as Sepharose Fast Flow (agarose beads crosslinked with epichlorohydrin, Amersham Biosciences, UK) or by means of epichlorohydrin activation of e.g. an agarose matrix such as Sepharose FF. Further preferred in combination with afore said preferred embodiment according to this paragraph is that the first ion exchanger is an anion exchanger, in particular a quaternary amine-based anion exchanger such as Sepharose Q™ FF (Amersham-Biosciences/Pharmacia), most preferably it is an anion exchanger having the functional exchanger group Q coupled to a matrix support which group Q is N,N,N-Trimethylamino-methyl, most preferably the anion exchanger is Sepharose Q™ FF from Pharmacia/Amersham Biosciences. The quaternary amino group is a strong exchanger which further is not susceptible to changes in pH of the loading/wash buffer. The fast flow exchanger matrix is based on 45-165 μm agarose beads having a high degree of crosslinking for higher physical stability; further sepharose is devoid of the charged, sulfated molecule fraction of natural agarose and does not allow for unspecific matrix adsorption of antibody, even under condition of high antibody loads. An example of such an embodiment can be found in the experimental section.

A contaminant protein A according to the present invention is any type of functional, IgG binding offspring of a protein A or a functional derivative thereof as defined above which is obtained upon eluting bound antibody from a protein A affinity chromatography column. Such contaminant protein A species may result e.g. from hydrolysis of peptide bonds which is very likely to occur by means of enzyme action in particular in industrial manufacturing. Protein A chromatography is applied as an early step in downstream processing when the crudely purified, fresh product solution still harbors considerable protease activity. Dying cells in the cell culture broth or cells disrupted in initial centrifugation or filtration steps are likely to have set free proteases; for regulatory purposes, supplementation of the cell culture broth with protease inhibitors prior or in the course of downstream processing is usually not accomplished, in contrast to biochemical research practice. Examples are Phenyl-methyl-sulfonyl-chloride (PMSF) or e-caproic acid. Such chemical agents are undesirable as an additives in the production of biopharmaceuticals. It is further possible that recombinant functional derivatives or fragments of protein A are less protease resistant than wild-type protein A, depending on the tertiary structure of the protein fold. Amino acid segments linking individual IgG binding domains might be exposed once the total number of binding domains is reduced. Interdomain contacts may possible contribute to the stability of domain folding. It might also be that binding of antibody by protein A or said functional derivatives thereof influences or facilitates susceptibility to protease action, due to conformational changes induced upon binding of the antibody. Again, wild-type or full length protein A or functional, engineered fragments thereof might behave differently. Preferably, contaminant protein A according to the present invention still is functional, IgG binding protein and thus is associated with the protein A-purified antibody when loaded onto the subsequent ion exchange separation medium according to the present invention. The high-affinity based association of contaminant protein A with the purified antibody is the reason why it is difficult to efficiently separate contaminant protein A from purified antibody.

Preferably, according to the present invention the antibody sought to be purified is harvested from a cell culture prior to purifying the antibody be means of protein A affinity chromatography. More preferably, said cell culture is a mammalian cell culture.

Mammalian cells have large compartments called lysosomes harboring degradating enyzmes which are disrupted upon cell death or harvest. In particular, said cell culture may be a myeloma cell culture such as e.g. NS0 cells (Galfre, G. and Milstein, C. Methods Enzymology, 1981, 73, 3). Myeloma cells are plasmacytoma cells, i.e. cells of lymphoid cell lineage. An exemplary NSO cell line is e.g. cell line ECACC No. 85110503, freely available from the European Collection of Cell Cultures (ECACC), Centre for Applied Microbiology & Research, Salisbury, Wiltshire SP4 0JG, United Kingdom. NS0 have been found able to give rise to extremely high product yields, in particular if used for production of recombinant antibodies. In return, NSO cells have been found to give reproducibly rise to much higher levels of contaminant protein A than other host cell types at least with certain protein A affinity chromatography systems employing recombinant, shortened fragments of wild-type protein A which recombinant protein A is possibly single-point attached protein A. An example of such is Streamline™ rProtein A affinity chromatography resin (Amersham Biosciences; essentially thioester single-point attached recombinant protein A as described in U.S. Pat. No. 6,399,750). Levels of about or in excess of 1000 ng contaminant protein A/mg antibody could be obtained with Streamline™ rProtein A affinity columns. The method of the present invention distinguishes from the prior art in efficiently reducing contaminant protein A from such elevated levels to <1 ng/mg antibody in a single, fast purification step, that is with a purification factor of about 1000×.

Further preferred is, alone or in combination with the preceding paragraph, that the antibody that is to be purified by means of protein A affinity chromatography is not treated as to inactivate proteases at or after harvest, more preferably is not in admixture with at least one exogenously supplemented protease inhibitor after harvest. In the present context, a protease inhibitor is any kind of chemical agent (which is not a protease) that is selectively inhibiting proteases whilst it does not chemically modify or do no harm to the tertiary and/or quaternary structure of the product protein, which may be e.g. an antibody; examples of proteinase inhibitors are chelators such as EDTA chelating metal ions important for the activity of metalloproteinases, may be considered such as well as N-[(2S,3R)-3-Amino-2-hydroxy-4-phenylbutyryl]-L-leucine Hydrochloride [Bestatin] which is equally active against metalloproteinases. Most preferably, said protease inhibitor is selected from the group consisting of PMSF and specific proteinase inhibiting peptides as described in Laskowski et al., 1980, Protein inhibitors of proteinases, Ann. Rev. Biochem. 49, 593-626. Examples are Leupeptin, Aprotinin for instance.

Operation of protein A affinity chromatography has been widely described in the technical literature and does not need to be further described. Another example apart from the above cited is e.g. Duhamel et al., J. Immunological Methods 31, (1979) 211-217, pH Gradient elution of human IgG1, IgG2 and IgG4 from protein A-Sepharose.

Of course exposure to acid pH conditions at about pH 3-4.5 upon elution, even when followed by immediate buffer exchange to about pH 7.5, will give inherently rise to aggregate formation. This problem has been perceived in the 1980's early on when protein A chromatography started to be widely available and was compared to the traditional chromatography trains. In a further preferred embodiment, acid elution from protein A matrix is followed by a virus inactivation treatment prior to loading of thus purified antibody the first ion exchanger, which virus inactivation treatment more preferably comprises low pH incubation at a of from pH 3.5 to pH 4.5 for about 50 to 90 min., preferably at a temperature of at least 30° C., more preferably of at least 45° C., or filtration through an animal virus reduction filter having a pore size of less than 1 μm, preferably less than 0.25 μm. Hence prior to reducing aggregate contents concomitant with removing contaminating protein A or protein a derivative, preferably a further important intervening step of treatment is done which ensues further aggregate formation and promotes aggregate growth; the treatment may be e.g. (thermal) challenge at acidic pH aiming at denaturing or de-assembling viral proteinaceous capsids or it may be an ultrafiltration step which suffers from denaturing membrane effects as well. Preferably, the virus reduction treatment is a low pH incubation step, easily allowing of a virus log reduction factor of about 6 to 8.

In one preferred embodiment, elution of antibody from the protein A chromatography column is done by using a low conductivity elution buffer of less than 5 mS/cm, preferably less than 3 mS/cm, more preferably less than 2 mS/cm, most preferably of about or less than 1.2 mS/cm of the buffer as it is prepared as a 1× buffer solution, prior to use in eluting the antibody product protein from the protein A column. Expediently, such buffer should likewise have a minimum conductivity of at least 0.1 mS/cm, preferably of at least 0.5 mS/cm, most preferably of a least 0.8 mS/cm. Surprisingly, in this aspect of the present invention, such low conductivity buffers, independent from the chemical nature of the buffer salt applied, proved consistently to show i. lowest aggregate contents immediately upon elution from the protein A column, ii. a most moderate increase of aggregate contents during a subsequent acid or low pH virus inactivation step (followed by immediate re-adjustment of the pH to about neutral pH, that is pH 6.5-7.5), and iii. still allowed of significant virus log reduction during acid pH treatment, typically giving a log reduction factor of about 7 after 60 min. exposure. This joint benefits of low conductivity buffers have not yet been appreciated.—Notably, at higher conductivitis (approx. 5-20 mS/cm), the nature of the buffer salt is strongly influence the increase in aggregate contents. Notably, citrate resulted in huge increase in the proportion of aggregates during acid pH virus inactivation step at such conductivities of 5-10 mS/cm and above.

Hence in a further preferred embodiment, alone or more preferably in combination with the afore described further embodiment of a low conductivity elution buffer, the protein A chromatography elution buffer employs as a buffering salt a monovalent carboxylic acid and/or its corresponding mono-carboxylate, e.g. its alkali or earth alkali carboxylate, having a pKa value of from pH 3 to 4, more preferably employs formate/formic acid. Optionally, it is further preferred that said mono-carboxylate or carboxylic acid is a monovalent a-amino acid which is devoid of any further charged groups in its side chain at pH 4, except for its H₄N⁺—CHR—COO⁻ head group with R being the side chain radical, is devoid of sulfhydryl functions and which amino acid preferably is water-soluble at pH 4 to a concentration of at least 5 mM, more preferably to at least 10 mM, and further preferably has a pKa value for its carboxylic acid function (pKa₁) of from pH 2 to 3. The amino acid may be a natural or non-natural amino acid, preferably is a natural amino acid. pKa value of the carboxylic head groups of natural amino acids may be found in Dawson et al, Data for Biochemical Research, 2^{nd} ed., pages 1-63, Oxford University Press (1969). More preferably, the amino acid is selected from the group consisting of glycine, alanine, a C1-C5 alkyl hydroxy amino acid such as e.g. serine or threonine or C1-C8 alkoxyalkyl or possibly polyoxyalkyl, amino acid. Glycine is strongly preferred for being used as a buffering amino acid for setting up the elution buffer for the protein A chromatography step according to the present invention.

Preferably, the contaminant protein A is reduced to a concentration of <10 ng/mg antibody, more preferably <4 ng/mg antibody, most preferably <1 ng/mg antibody in the flow-through of the first ion-exchanger, wherein antibody is preferably to be understood as to refer to IgG. The Elisa assay method for validation of these threshold values is described in detail in the experimental section; it should be noted that acidification of the sample to a pH=4, preferably in the presence of a mild detergent, is crucial for accurate determination of the amount of leaked protein A. It goes without saying that his is threshold is to be understood such as that the loading capacity of the first ion-exchanger for protein A binding is never exceeded, leading inevitably to break-through of contaminant protein A.

A suitable Elisa-based method for assaying protein A or protein A fragments is described in U.S. Pat. No. 4,983,722. Suitable anti-protein A antibodies are commercially available, e.g. from Sigma-Aldrich. In particular when using derivatives of protein A which derivatives have been engineered to harbor additional sulfhydryl groups, proper maintenance of the protein standard is important. It may be important to verify the monomeric character of such pure protein A derivative used as a standard for quantification of the test sample, since covalent di- or multimers formed via —S—S— bridges could lead to wrong results. Verification can be easily achieved by SDS-PAGE analysis under reducing and non-reducing conditions, as is customary in the art. Reduction of such protein A derivative-standard solution by means of DTT or beta-mercaptoethanol helps accordingly to circumvent errors of measurement in the ELISA-technique.

Further preferred, in the method according to the present invention at least 70%, more preferably at least 80%, most preferably at least 90% of the antibody loaded onto the first ion exchanger can be recovered in the flow-through of the ion-exchanger. Preferably, and disregarding glycoforms and eventual processing variants of the same antibody, there is only one type of antibody at the species level present in the starting mixture that is going to be purified by means of protein A affinity and subsequent ion exchange chromatography according to the present invention. For instance, when purifying a human or human-mouse chimeric or primate or primatized IgG antibody according to the present invention, no bovine IgG as would be carried over from serum in serum-supplemented cell culture is present. To put it differently, preferably the method of the present invention is applied to curde, unpurified antibody harvested from serum-free cell culture.

The first ion exchanger according to the present invention is an anion exchanger resin; protein A can be bound by both types of resin as described (EP-289 129 B1). The first ion exchanger or anion exchanger can be operated in the column mode at a certain flow rate or in batch operation mode, by submerging the ion exchange resin into the mildly agitated sample solution and further exchanging liquid media by filtration subsequently. According to the present invention and taking into account the pI of a given antibody, it is possible to define suitable conditions of pH and ionic strength for loading the first ion exchanger, which conditions result in retaining the antibody in the flow through whilst the protein A contaminant is bound and thus removed from the antibody. As has been said before, the method according to the present invention allows of faster separation of antibody from contaminant protein A. Examples of functional groups of such first, anion exchanger that are attached to a matrix support are e.g. primary, secondary, and particularly tertiary or quaternary amino groups such as aminoethyl, diethylaminoethyl, trimethylaminoethyl, trimethylaminomethyl and diethyl-(2-hydroxypropyl)-aminoethyl. Suitable chromatographic support matrixes for the anion exchanger are known in the art. Examples are agarose-based resins and beads, dextran beads, polystyrene beads and polystyrene/divinyl-benzene resins. It is further equally possible to use ion exchange membrane absorbers (e.g. Sartobind Q from Sartorius). For the obvious purpose of so allowing of higher flow rates and shorter separation times, the matrix material may a perfusion material which is a further preferred embodiment. It may be made up from perfusion-proficient beaded matrix material (cp. e.g. Afeyan et al., 1991, J. Chromatography, 544, 267.-279), including ceramic matrices, or may be a monolithic perfusion material such as the SepraSorb® branded fast flow material sold by Sepragen Inc. (Hayward, Calif./U.S.A.). SepraSorb® was developed specifically as an alternative to the beaded matrices. It is a cross-linked, sponge-like, regenerated cellulose material with a continuous, interconnected, open pore (50-300 micron) structure. This monolithic matrix has readily accessible surfaces on to which the ion exchange functional groups (DEAE, QM, CM & SE) are easily immobilized. Feed stream liquids actually flow perfusion-like through the interconnecting pores of the continuous matrix, as opposed to around the beads as in conventional media. SepraSorbâ provides many advantages over beaded media, in production scale. It can easily accommodate flow rates of 100 ml/min with back pressures rarely exceeding 1 bar (14.5 psi). A monolithic matrix is very easy to handle and to configure avoiding cumbersome and time consuming column packing. The matrix avoids clogging, channeling and is resistant to cracking, hence allows of extended operation time and number of operating cycles.

Most preferably, the ion exchanger is a quaternary amine-based anion exchanger mounted on an agarose matrix such as e.g. Sepharose CL-6B or Sepharose Fast Flow (FF) from Amersham-Biosciences/Pharmacia. An example of such is Sepharose Q™ from Amersham-Biosciences/Pharmacia. Further preferred in conjunction with the use of a first anion exchanger is that the antibody according to the present invention is a monoclonal antibody that has an isoelectric point (pI) which is at least two pH units above, that is it is more basic than, the pI of the protein A used in the preceding protein A affinity chromatography step; e.g. whereas native protein A has a pI of about 5.0, Streamline recombinant protein A has a pI of about 4.5. Preferably, the antibody according to the present invention is a monoclonal antibody that has an isoelectric point (pI) which is at least 6.5 or above, more preferably is 7.0 or above, most preferably has an pI of at least 7.5 or above. It should be noted that this refers to the pI of the actually harvested and purified antibody, not the pI that can be simply predicted from the amino acid sequence alone. The actually purified antibody molecule may have undergone further modifications of the polypeptide backbone such as glycosylation, which modifications may add charged to moieties and thus may have changed the pI of the molecule. Upon determination of pI for product antibody by means of isoelectric focusing (IEF), the microheterogenity of posttranslational processing of the antibody protein, e.g. a monoclonal antibody protein, leads to a wider pI-range for individual glycoforms of product antibody, the totality of which resembling to a smear in an IEF gel rather than a single band and thus a specific numeric value for at least the majority of product. According to the present invention, in such afore mentioned preferred embodiment, the ‘pI of an antibody’ refers to that share of antibody product molecules whose pI is within the preferred range of pI as specified above. All further definitions of this description, such as the %-proportion of antibody recovered after a given purification step, refer to said pI-compliant share of antibody only. Further preferred is that in approximation, the numeric mean pI value of the ‘smear’ range as determinable by experiment is to be construed as the pI or average pI according to the present invention, presuming this being a reasonably fair representation of the quantitative distribution of glycoforms.

Preferably, for the joint purpose of removing both aggregate and contaminating protein A or protein A derivative, the pH of buffer used for loading and rinsing the first ion exchanger is set as to avoid in principle straightforward repulsion in between the charged groups of the ion exchange material when exposed to the buffer and both the protein A or protein A contaminant and the antibody to be purified. Given the purpose of enabling static binding of the protein A species to the ion exchanger under the buffer conditions applied, whilst allowing likewise of non-binding of the antibody under the same buffer conditions, it ensues that taking the pI's of antibody and protein A or protein A derivative into account, the first ion exchanger will normally be an anion exchanger to be operated at a pH close to or above the pI of the antibody sought to be purified. Hence the antibody's surface charge is either zero or is negative, but is never bluntly positive and hence repelling. Suitable adjustment of ionic strength is then vital to achieve non-binding conditions for the antibody whilst protein A is bound. However, this does pertain to average pI value as defined above; hence having regard to glycoforms of antibody, this doesn't mean that a smaller share of glycoforms might be not be close or be at pI. Further, in view of aggregate removal, it is to be contemplated that resolution of monomer from aggregates occurs in a flow-through mode but owes part of effect at least to transient and weak ionic attraction interaction with the ion exchange in a non-binding mode; monomer and aggregates will display subtle differences in surface charge and hence pI; therefore it is possible to work the method of the present invention at least for some antibodies successfully even e.g. in the range of an buffer pH up to 0.5 pH units below the antibody's pI with an anion exchanger (and vice versa 0.5 pH units above an antibody's pI when working with a cation exchanger in view of aggregate removal only, s. below) since, explaining the phenomenon with hindsight, only the monomer but not the aggregates having different accessible surface and eventually neutral or even negative charge is actively repelled by ionic forces from the ion exchanger material. However, this embodiment of working the present invention would consequently be highly dependent on the aggregates pI and hence aggregation properties, which is not predictable, hence it may not be expected with any given antibody. According to another preferred embodiment, it is less desirable to use a buffer pH set at the average pI of the antibody to be purified in view of optimum separation from contaminant protein A and aggregate; preferably, the buffer pH for loading and rinsing the first anion exchanger giving rise to the flow-through that is collected and in harboring the antibody product peak under the non-binding chromatography conditions according to the present invention is set a pH above the pI, more preferably is set at a pH of pI+0.5 pH unit, of the antibody monomer.

The mode of operation of a first anion exchanger according to the present invention requires buffer exchange of the acidic or neutralized eluate from the protein A affinity chromatography step with the equilibration buffer of the first anion exchanger. Equilibration buffer and loading buffer are identical in the method of the present invention. Commonly employed ultrafiltration devices such as sold by Amicon or Millipore can be expediently used for that purpose; those avoid the dilution effects whilst using e.g. low molecular weight porous filtration matrices such as Sephadex G-25. The equilibration buffer according to the present invention preferably has a salt concentration of a displacer salt such as e.g. sodium chloride in the range of 1 to 150 mM, more preferably of from 5 to 110 mM, most preferably of from 20 to 100 mM salt. The pH of the equilibration buffer is preferably in the range of pH 6.5 to pH 9.0, more preferably is in the range of pH 7.5 to pH 8.5, most preferably is in the range of pH 7.9 to pH 8.4. It should be kept in mind that N-terminal amino function of a protein has a pKs value of about 9.25, thus binding of contaminant protein A and any other already negatively charged protein to an anion exchanger will get stronger at more basic pH; for a given application, the pH of the loading buffer might need finetuning for optimal discrimination of binding and non-binding for a given pair of antibody and contaminant protein A having differing pI values and different content of cysteine and histidine side chains which may contribute to changes in charge within the selected ranges of pH. Further, a more basic pH interferes with proteinA-antibody interactions as will do any increase in ionic strength; likewise, ionic strength needs finetuning to balance prevention of binding of antibody with the need to abolish binding of contaminant protein A. It goes without saying for the skilled artisan that the ionic strength of the buffer is usually inversely correlated with the pH value; the more strongly protein A gets bound to the anion exchanger depending on pH, the more salt is tolerated for preventing binding of antibody and for interfering with potential proteinA-antibody interactions. Thus, the above given ranges for pH and displacer salt thus are to be understood as to be correlated: The lower the pH, the less salt is found permissible within the above given preferred ranges for working the invention. Further salt freight is added by the pH buffering substance, further increasing the ionic strength of the solution. The ionic strength can be determined by measuring the conductivity of the equilibration buffer. The term ‘conductivity’ refers to the ability of an aqueous solution to conduct an electric current between two electrodes measures the total amount of ions further taking charge and ion motility into account. Therefore, with an increasing amount of ions present in the aqueous solution, the solution will have a higher conductivity. The unit of measurement for conductivity is mS/cm (milliSiemens/cm), and can be measured using a commercially available conductivity meter, e.g. from Topac Inc. (Hingham, Mass./U.S.A.) or Honeywell. In the context of the present application, all numerical values pertain to the specific conductivity at 25° C. Preferably, the loading or equilibration buffer for the first anion exchange step has a conductivity 0.5-5 mS/cm, more preferably of from 1-3 mS/cm, most preferably of from 1.25-2.5 mS/cm. Ideally, it has a conductivity of about 2 mS/cm.

Examples of suitable buffer salts can be found in Good, N. E. (1986, Biochemistry 5:467-476). E.g. Tris.HCl buffer or a sodium hydrogen phosphate buffer as customarily employed are suitable buffering substances. The concentration of the buffer substance is customarily in the range of e.g. 10-40 mM buffer salt. Amongst potential anion species useful for devising a buffer, those having lower specific strength of anion elution as compared to chloride, which property of low elution strength is approximately inversely correlated with the density of ionic charge and is approximately proportional to the ionic size, are preferred. Empirical comparisons of strength of anionic elution are tabulated in the standard textbooks of biochemistry. More preferably, the buffer substance according to the present invention is a phosphate buffer. Hydrogenphosphate has a low elution strength, in particular if employed at a pH at or below pH 8, and further excels by particularly low chaotropic properties.

In a further preferred embodiment, the first anion exchanger is a ceramic matrix-anion exchanger such as the Biosepra-branded HyperD® anion exchangers, more preferably a ceramic matrix-anion exchanger having a quaternary ammonium (=quaternary amine-based) ionic, matrix bonded functional group. These are extremely useful for purification at a therapeutic scale. Most preferably, the quaternary, ceramic anion exchanger is a Q-ceramic matrix anion exchanger such as, and particularly preferred, the Q-HyperD® anion exchanger resin sold by Ciphergen Biosystems Ltd., Guildford/Surrey, UK under the ‘Biosepra’ trademark. The above and below mentioned preferred embodiments on pI of antibody, protein load and buffer pH are also preferred in combination with this embodiment, with the exception of the preferred conductivity of buffer when using Q-Hyper D® material being at best 0.5-2 mS/cm, more preferably being in the range of 0.6-1.7 mS/cm, most preferably at about 1 to 1.5 mS/cm and in particular when using Q-Hyper D®-F ion exchanger. This conductivity ensures best purification result in view of deriching contaminant protein A or fragments thereof from the product protein. The Ceramic HYPERD sorbents are made using a rigid porous bead, which is coated and permeated with a functionalized hydrogel. This gives the beads outstanding rigidity and flow performance, as well as exceptional mass transfer and dynamic properties. The Ceramic HYPERD sorbents are very easy to use. Their relatively high density makes them easy to pack and use in very large columns. The complete lack of shrinking or swelling eliminates the need for repeated packing/unpacking of columns. Today, columns in excess of 500 liters are used for preparative chromatography of molecules for therapeutic use. The Ceramic HYPERD ion exchangers are also available in a 50 μm grade (F grade) for preparative processes, with their high capacity and lower back pressure the 50 grade is perfect for capture processes and general downstream processing. The ceramic nature of the bead makes it chemically very stable and it can be cleaned using most commonly used cleaning agents, including 0.5 M NaOH.

The above set forth conditions are setting the framework for allowing of contaminant A removal in a flow-through mode. For further concomitant removal of aggregate from a given antibody monomer, further careful testing within the generic ranges given above for conductivity, pH and identity of buffer salts etc. is required for defining admissible conditions which are allowing of both simultaneous protein A removal AND aggregate resolution for a given antibody. As said before, this will be highly specific for a given antibody and may not be defined any further in generic terms. Studies further exemplified in the experimental section have shown that operation of anion exchange matrices in a non-binding mode result in fractionation of aggregates and monomer such that the aggregates are resolved mainly on the down slope of the unbound protein peak fraction of the antibody in the flow-through. This surprising finding applies to product protein monomer purification beyond just antibodies, leaving the protein A aspect aside. By careful selection and pooling of fractions, the level of aggregates can be reduced in the main elution peak of the flow through of the first ion exchanger. No precedent for such finding has ever been reported in the scientific literature, nor could it have been anticipated due to the fact that the flow through-mobile phase is just not expected to interact significantly with the solid phase, that is the ion exchange material. Most astounding, the aggregate tailing effect in the flow through takes place at a buffer pH for the flow-through buffer liquid that is far off from the average pI of the product protein or antibody monomer and that chargewise allows of ionic attraction at ionic strength impermissible to static binding. Both anion and cation exchangers have been found to allow of aggregate resolution or tailing in the flow-through fractions in this way. Speculative and without being bound to such explanation, one might suppose in hindsight that at least partially some weak but dynamic ionic attraction with ion exchanger contributes to this effect, as has been said before. Further contributions might be made by the matrix support material. In one temptatively preferred embodiment for achieving separation of aggregate from product protein monomer or antibody monomer in a flow-through mode on an ion exchange resin according to the present invention, the matrix material of the first anion exchanger is a polymeric polyol or polysaccharide. Avidity aspects of the alleged resolution effect being amplified by redundancy of binding sites on the same molecule, the larger the aggregate is, may contribute to this. Still then, dimeric aggregates such as made up from two IgG antibodies can be successfully separated from monomeric IgG antibody according to the method of the present invention.

Whereas batch mode operation is possible, column operation mode is preferred for the first anion exchanger step. In that case, a flow rate of about 10 to 60 ml/h can be advantageously employed. The loading concentration of antibody loaded can favorably be in the range of 10 to 30 mg antibody/ml exchange resin. It goes without saying that the use of extremely diluted samples would give rise to decreased yield of antibody, as is known to the skilled person. The antibody sought to be purified is collected in the low-through of the loading operation including about one column volume of wash with the same equilibration buffer. The pH of the flow-through may be adjusted to neutral pH for improving stability and preventing new aggregation and/or precipitation of antibody protein.

On a general note, the method of the present invention can not be exploited for antibodies that have been raised against protein A-borne epitopes. Such antibodies are disclaimed, though this is an obvious limitation to the skilled artisan. It is further to be noted that the meaning of a ‘first’ ion exchange chromatography step according to the present invention, is an open definition and has only regard to the chronology of events according to the present invention; it is not to be construed as to exclude any intervening ion exchange chromatography step that is conducted in the traditional binding and elute mode as regards the protein or antibody protein, respectively, that is sought to be purified.

The most appealing feature of the method of the present invention is that purifying antibody via an anion exchanger in a non-binding or flow-through mode, the capacity of the column is not all limiting the through-put of material; the capacity is only decisive with regard to minor amounts of contaminant protein A retain. This saves a lot of processing time and material resources whilst allowing for very efficient removal of protein A contaminant.

An afore mentioned further object of the present invention that has partially already been alluded to is a general method for removing protein aggregates from monomers of a product protein to be purified, comprising the steps of comprising the steps of firstly, loading a solution comprising product protein which product protein comprises monomeric and aggregated forms of said protein onto an ion exchange material under conditions which allow of resolution in the flow-through, by means of fractionation of the flow-through, of said product protein aggregates from said product protein monomer which monomer preferably is not further complexed with a second protein ligand, and secondly further fractionating the flow-through and harvesting from the flow-through of the ion exchanger at least one product protein monomer fraction having reduced contents of product protein aggregate as compared to the composition of product protein loaded onto the ion exchange material for purification.

The foregoing definitions apply here, too, in particular those for practical conduct of the flow-through ion exchange chromatography; hence the aggregate is accordingly to be understood as to be a non-specific dimeric or higher order, soluble aggregate of a given protein which protein may comprise single or multiple, covalently bonded protein chains. Preferably, the aggregate comprises both dimers and higher order aggregates of the same product protein, as has already been defined above for the specific example of an antibody and exemplified for an IgG, and all such types of aggregates as defined are found to be deriched by the ion exchange chromatography step which according to the present invention are carried out in a flow-through mode. Both anion and cation exchange are found working the method of the present invention; more astounding, the method is found working both at the pI of the product protein monomer sought to be purified as well as at an pH of buffer leading to ionic attraction in between the product protein monomer and the ion exchange material due (attraction of e.g. positive charges both on the exchanger and the protein surface), though not leading to productive binding due to buffer conductivity being non-permissive for product protein becoming bound to the ion exchanger. In short, for achieving aggregate removal, when using a cation exchanger in a non-binding mode with regard to the product protein sought to be purified, the cation exchanger should be worked but with a loading and rinsing (post-loading) buffer having a pH at about or below the average pI of product protein, vice versa, when using an anion exchanger, the anion exchanger should be worked solely with one or several loading and rinsing (post-loading) buffer having a pH at about or above the average pI of product protein. Preferably, the buffer pH is not set at the pI of the product protein, as explained in the foregoing already in the context of antibody purification but with general meaning. The explanations made above on pI of glycoproteins and glycoform distribution and experimental determination of pI apply likewise to the present object. It goes without saying that said rinsing and loading buffer must be compliant with establishing non-binding mode of operation for a given product protein monomer and within this limitation being set, said rinsing buffer might be same or different from the loading buffer as regards composition or that even several different rinsing buffers could be used successively, though not distinct benefit of doing so is perceivable. For sake of simplicity, sample preparation loading and rinsing are e.g. conducted with the ever same buffer being used. However, more intricate modes of loading than just pouring liquid sample preparation suspended in buffer admissible with non-binding mode operation the sample onto a homogenous ion exchange column may be perceived for conducting the chromatographic purification method of the present invention.

Preferably, fractionation is achieved by fractionating or splitting the antibody peak of the flow-through into at least two fractions and wasting the tail fraction. In this way, monomericity of the antibody harvested can be set to amount to a purity of at least to 99% monomer based on total product protein content whilst substituting tedious gel permeation or size exclusion chromatography methodology or equally low-throughput, sophisticated machinery based, expensive split-flow or sedimentation techniques with the most widely applied, high-throughput and extremely fast ion exchange chromatography—to the same end. There is no faster processing than by collecting directly the flow-through of an ion exchange column, without conducting any further tedious washing, elution and regeneration steps.

### EXPERIMENTS

### 1. Protein A Elisa

Numerous Elisas for testing of protein A or recombinant protein A have been described (see U.S. Pat. No. 4,983,722 and references described in there). For all work described below, a simple sandwich Elisa was used in which capture anti-protein A antibody coated on a flat-bottomed 96 well microtiter plate (Nunc™) retains the protein A. Bound protein A is then detected an a biotinylated anti-protein A detection antibody, which allows for further binding of streptavidin conjugated horseradish peroxidase (Amersham #RPN 1231). Commercially available anti-protein A rabbit antibody (raised against the natural S. aureus protein A) for capture is available from Sigma-Aldrich (#P-3775). It was this antibody which was used through-out this study. The detection rabbit antibody was equally purchased from Sigma-Aldrich (#3775). After coating the protein by unspecific adsorption process, the coated protein is used to retain protein A-specific protein A capture antibody which capture antibody is further detected with biotinylated rabbit anti-protein A and streptavidin-horseradish peroxidase. Tetramethyl benzidine is used as the chromogenic substrate. Samples of unknown concentration are read off against a standard curve using the very parent-protein A or -protein A derivative of the contaminant protein A sought to be detected. Coating at acidic pH as well as proper preparation of the standard has proven important. In particular for recombinant protein A's engineered to carry additional cysteine residue such as e.g. Streamline protein A™ (Amersham Biosciences, formerly Pharmacia), the standard solution was found to require pretreatment with a reducing sulfhydryl agent to ensure monomeric state of the protein standard solution.

Wild-type protein A standard, in contrast, is commercially available from a number of companies, e.g. Sigma-Aldrich/Switzerland (#P6031) or Pharmacia (#17-0770-01) and does not require such pretreatment. For the below described experiments observing leakage of contaminant protein A from Streamline™ matrix, samples of unconjugated recombinant protein A obtained from the manufacturer were used as a standard.

### 1.1 Pretreatment of Cys-Enriched Protein A-Standard

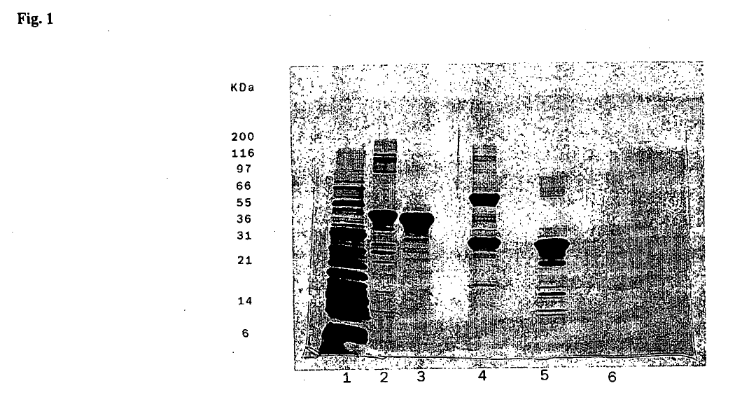

Pure recombinant protein A-Cys as commercially in the Streamline™ protein A affinity chromatography (Amersham Biosciences) column material was obtained freeze-dried from Pharmacia/Amersham Biosciences. Up to 20 mg/ml protein were dissolved in 0.1M Tris pH 8 containing 0.5 M NaCl, 1 mM EDTA and 20 mM dithioerythritol, incubated for 15-30 min. at room temperature and desalted with a disposable PD-10 gel filtration column (Amersham Biosciences). All buffers used for handling the standard solution before coating should be N₂-treated to prevent oxidation of the thiol groups. Preparation of the protein standard was carried out at best immediately prior to use of the standard for coating the microtiter plates. Optionally, a 1 mg/ml stock solution was prepared and kept at −65° C. in a freezer, after thawing, monomeric character of protein A was assayed from an aliquot loaded on non-reducing SDS-PAGE. The concentration of protein standard was determined by Bradford assay (Bradford et al., 1976, Anal. Biochem. 72:248-254; Splittgerber et al., 1989, Anal. Biochem. 179:198-201) as well as by automated amino acid analysis. The result of such pretreatment is shown in FIG. 1 by means of non-reducing 10% SDS-PAGE for a staphylococcal protein A standard (lane 1: native protein A; lane 2: after pretreatment) and pure, uncoupled Streamline™ recombinant protein A (provided by courtesy of Pharmacia, now Amersham-Biosciences; lane 4: native recombinant protein A; lane 5: after pretreatment). Lane 1 is a molecular weight marker with the corresponding molecular masses being denoted on the vertical axis. The recombinant protein A from Pharmacia harboring an additional Cys residue shifts after reduction to lower molecular weight; a monomeric band at about 34 kD is preserved and much more intense, stemming obviously from dissociation of disulfide bridged dimers.

### 1.2 Elisa

### 1.2.1 Preparation of Sample

By two dilution steps, 1: 200.000 dilution of the 1 mg/ml protein A standard stock solution was prepared to provide the top standard at 50 ng/ml. Thereof, dilutions down to 0.2 ng/ml were prepared for assaying the standard curve. Further, the dilutions of the standard (‘spinking solutions’) were used for spiking of duplicate product samples to be tested in order to exclude presence of interfering substances in the sample.

For final product sample testing, every sample is divided into 2 equal volumes of 500 μl. One is spiked with the 1000 ng/ml spiking solution, or the 10 μg/ml solution if appropriate, to give a final protein A content of 10 ng protein A per mg of antibody. The other half is spiked with the same volume of sample buffer; thus the dilution factor of the product sample due to spiking is accounted for. Both types of preparation will be referred to as ‘spiked sample’ in the following. The sample buffer was made up from 7.51 g Glycine (base), 5.84 g NaCl, 0.5 ml Triton X-100 to a volume of 1 L with deionized or bidestillated water.

For optimal accuracy measurements, the antibody concentrations in the samples were pre-determined by customary Elisa's well known in the art. A further standard solution was spiked with an equal amount of a known standard antibody of comparable constant region affinity for protein A, to determine efficiency of the acidification step and to unravel any potential systematic error introduced by antibody binding to and thus scavenging protein A from capture in the assay.

Acidification: To 450 μl of spiked sample or standard is added 200 μl of 0.2 M citrate/0.05% Triton X-100 buffer at pH 3.0. All samples were done in triplicate. Further, dilutions of sample were prepared and tested in triplicate since the assay works optimal for antibody concentrations being in the range of 1 mg/ml and 0.2 mg/ml. The acidification step is crucial in the present assay to liberate contaminant protein A or A fragments which were otherwise bound to the excess of antibody present in the sample solution.

1.2.2 Coating of Microtiter Plates with Antibody

Coating buffer was made up from 1.59 g/L Na2CO3, 2.93 g/L NaHCO3 and 0.20 g/L sodium azide. The pH of the buffer was adjusted to pH 9.6. Add 100 μl antibody solution per well comprising antibody in an amount sufficient as not to show saturation for the protein A standard. Cover plate with plastic film and place in humidity chamber. Incubate at 37° C. overnight for approximately 18 hours. Rinse all wells 3 times with at least 300 μl washing buffer [NaCl 5.8 g/L, Na₂HPO₄ 1.15 g/L, NaH₂PO.H₂0 0.26 g/L, EDTA 3.7 g/L, Tween-20 0.2 g/L, butanol 10 ml/L, pH 7.2], and tap dry. Add 250 μl blocking buffer [coating buffer with 0.5% casein hammarsten] to each well and incubate for 2 hours at ambient temperature on a benchtop orbital shaker (speed 120 rpm). Rinse all wells three times with at least 300 μl washing buffer, and tap dry.

### 1.2.3 Incubation of Sample and Detection

Plate out standards and samples including any spiked samples with 100 μl/well. Cover plate with plastic film and incubate for 90 minutes at ambient temperature on an orbital benchtop shaker. Rinse all wells three times with at least 300 μl washing buffer, and tap dry. Dilute biotinylated rabbit anti-protein A at the previously determined optimal dilution. Add 100 μl/well, cover plate with plastic film and incubate for 90 minutes at ambient temperature on an orbital shaker. Repeat rinsing.

Dilute strepavidin-horseradish peroxidase at the previously-determined optimal dilution using conjugate buffer [Na2HPO4 1.15 g/L, NaCl 5.84 g/L, NaH2PO4.H20 0.26 g/L, EDTA 3.73 g/L, Triton X-100 0.05% (v/v), pH 7.2]. Add 100 μl/well, cover plate in plastic film and incubate for 45 minutes at ambient temperature on an orbital shaker. Repeat rinsing. Add 100 μl freshly-prepared tetramethyl-benzidine (TMB, ICN product number #980502) substrate solution. The substrate solution is prepared like this: A stock solution is prepared by dissolving 10 mg TMB in 1 ml DMSO. 10 μl of that stock, further 10 μl of H₂O₂ are added to a 2.05% (w/w) sodium acetate aqueous solution that was adjusted to pH 6.0 with 0.5 M citric acid. It goes without saying that all water used for preparing any reagent of the assay is of highest quality, that is deionized ultrapure or at least bidestillated water.

The substrate solution is incubated at ambient temperature for 8-11 minutes on a shaker. The reaction is then stopped by adding 50 μl per well of stopping solution [13% H₂SO₄]. Within 10 min. after addition of the stopping solution, the absorbance of the wells at wavelength 450 nm is determined on a plate-reading spectrophotometer.

The detection limit for such Elisa is 0.2 ng/ml Protein A, with a working range of from 0.2 to 50 ng/ml. The interassay variability is less than 10%.

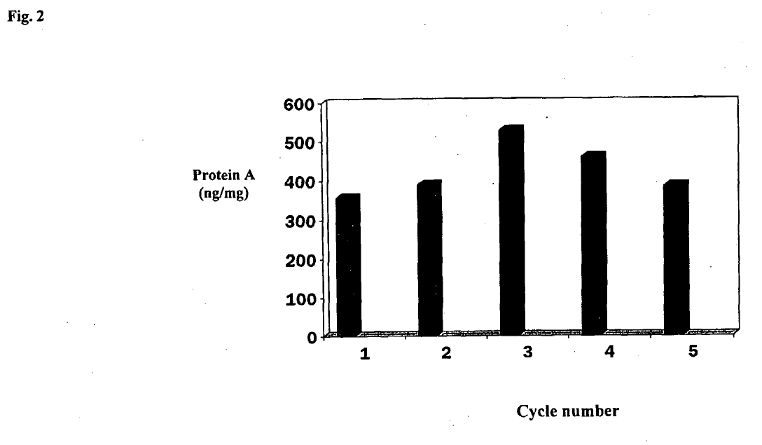

FIG. 2 shows the levels of leaked recombinant protein A in antibody eluates from Streamline™ recombinant protein A chromatography with single-point attached protein A in thioether linkage. The cycle number refers to repeated use after elution with 1 M sodium chloride and re-equilibration. Whereas leakage from cell culture broth from hybridoma cell culture was typically in the order of 500 ppm, other cell types gave levels as high as 1000 ppm. An overview on the rate of leakage from differently sourced matrices is given in Table 1; chromatography was performed according to manufacturer' instruction.

TABLE 1

| | | | | Working | |
|---|---|---|---|---|---|
| | | Coupling | Typical leakage | Capacity | Flow Rate |
| Matrix | Supplier | chemistry | p.p.m | (mgml⁻¹) | (cmh⁻¹) |
| Native Protein A | Amersham- | Multi -point | 10-20 | 5-20 | 30-300 |
| Sepharose 4FF | Biosciences | attached | | | |
| | | CNBr | | | |
| rmp Protein A | Amersham- | Multi point | 10-20 | 5-20 | 30-300 |
| Sepharose | Biosciences | attached | | | |
| Poros A High | Applied | Multi point | 10-50 | 10 | 500-1000 |
| Capacity | Biosystems | attached | | | |
| Protein A | Biosepra | Multi point | Up to 300 | 10-20 | 200-500 |
| Ceramic HyperD | | attached | | | |
| rProtein A | Amersham- | Single point | 50-1000 | 20-40 | 30-300 |
| Sepharose | Biosciences | attached | | | |
| | | Thioether | | | |
| | | linkage | | | |
| MabSelect | Amersham- | Single point | 50-1000 | 20-40 | 500 |
| | Biosciences | attached | | | |
| | | Thioether | | | |
| | | linkage | | | |
| STREAMLINE | Amersham- | Single point | 50 -1000 | 20-40 | 200-400 |
| rProtein A | Biosciences | attached | | | |
| | | Thioether | | | |
| | | linkage | | | |

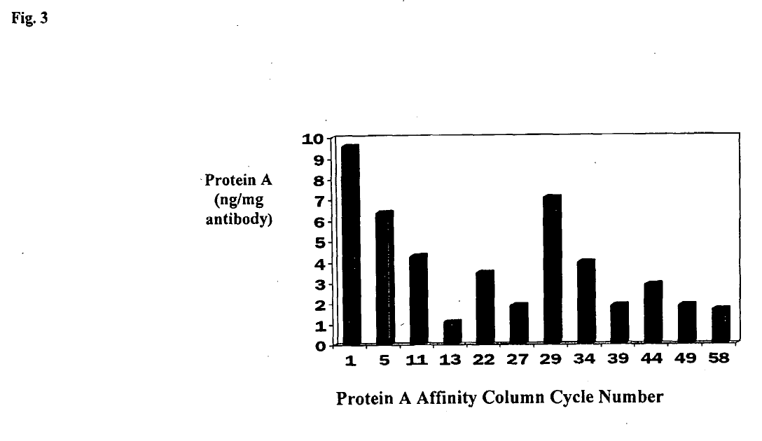

FIG. 3 further provides data on insubstantially reduced leakage of contaminant protein A during repeated runs of the protein A affinity chromatography with the same affinity matrix material; wild-type protein A multipoint-attached Sepharose 4 FF (Amersham-Biosciences) was repeatedly used as described in section 2.1 below and the level of contaminant protein A in the eluate, before any further processing of eluate, was determined by Elisa as described above.

### 2. Protein A and Sepharose Q Chromatography/without Concomitant Fractionation for Aggregate Derichment

2.1 Protein A Affinity Chromatography with Streamline™

Cell culture supernatant from a NS0 myeloma cell culture was crudely purified by centrifugation and in depth filtration and concentrated by ultrafiltration; ultrafiltration was also used to exchange the culture fluid to PBS pH 7.5. The titer of the antibody #5 produced by the cells was 0.2 mg/ml, a total of 1 L buffer-exchanged supernatant was loaded. The pI of the monoclonal antibody #5 was 8.5. The protein A Streamline™ column (5.0 ml volume) was previously equilibrated with 10 column volumes of 50 mM glycine/glycinate pH 8.8, 4.3 M NaCl; flow rate was at 200 cm/h. For loading, the column was operated at a flow rate of 50 cmh⁻¹; loading capacity was about 20 mg/ml matrix material). Before elution, the column was washed with at least 10 column volumes of glycine equilibration buffer supplemented with additional 200 mM NaCl and 0.1% Tween-20. Elution was achieved with elution buffer made up of 0.1 M glycine/HCl pH 4.0 buffer. Immediately after elution, fractions of eluate comprising the antibody peak were neutralized with an adequate aliquot of 0.5 M Tris HCl pH 7.5 and buffer exchanged with an Amicon diafiltration device with loading/equilibration buffer (10 mM Tris/HCl pH 8.0, 50 mM NaCl) of the present invention for the subsequent anion exchanger step for preventing longer exposure to acidic pH.

The antibody concentration and the contaminant protein A concentration were determined as described above. The level of contaminant protein A in the eluate amounted to 1434 ng/mg antibody before and amounted to 1650 ng/mg antibody after diafiltration. The recovery of antibody based on the titer of the buffer exchanged supernatant solution prior to loading was 81%; the concentration of antibody in the diafiltrated solution was 3.6 mg/ml.

### 2.2 Q-Sepharose FF Anion Exchange Step in Non-Binding Mode

The purified antibody from section 2.1 was further processed as described: A 5.0 ml Q-Sepharose FF column (Amersham-Biosciences) was packed 10 ml of 0.1 M NaOH, followed by 2 column volumes of 0.1 M Tris pH 8, and equilibrated in 10 column volumes of 10 mM Tris pH 8/50 mM NaCl, at a flow rate of 75 cm/h. After equilibration, the flow rate was reduced to 50 cm/h. 6 ml of the diafiltrated antibody solution was loaded onto the column and the flow-through was collected for further processing; the flow-through was continued to be collected until, after having loaded the column with the initial 6 ml and having continued thereafter with pure loading or equilibration buffer 10 mM Tris pH 8, 50 mM NaCl, the absorption of the flow-through monitored at 280 nm was back to baseline.

The total recovery of antibody in the flow-through was 23 mg antibody (87%). Determination of the level of contaminant protein A resulted in <3 ng/mg antibody. Further processing of this Q-Sepharose purified antibody batch by gel filtration (size exclusion chromatography, SEC) over Sephacryl S-300 in 10 mM Phosphate pH 7.0, 140 mM NaCl buffer at a flow rate of 10 cm/h with a loading ratio of 15 mg antibody per ml gel was found not change this trace contaminant protein A level substantially any more. By experience, SEC may be used to further reduce levels of about 30-100 ng/mg contaminant protein A to about 1-5 ng/mg. Thus SEC has a very low purification factor with regard to trace amounts of protein A, possibly accounting for affinity interactions in between antibody and contaminant A. However, due to the unavoidable dilution of sample and slow processing with allows for same decay of the antibody protein, SEC will allow for 70% recovery only of the amount of antibody loaded. This means SEC will unavoidably result in loss of material whilst requiring much time.

The Q-Sepharose column was recycled for further use by separate elution in 2M NaCl and further equilibration as described above.

2.3 Streamline™ Protein A Affinity Chromatography with Custom-Made, Multipoint-Attached protein A

This multipoint-attached Streamline™ protein A-affinity matrix was custom made and supplied by Pharmacia Biotech (now Amersham-Pharmacia). It was made up by the manufacturer by coupling the same 34 kD Streamline™-type recombinant protein A having a terminal Cys residue to the same Sepharose matrix material, but used traditional CNBr chemistry for activation and coupling instead of epoxide-mediated activation and selective reaction conditions for coupling of —SH groups only (see product information from manufacturer). The method of exp. 2.1 was repeated and the level of contaminant protein A was determined with 353 ng/mg antibody. Hence it may be inferred that the mode of coupling of the protein A to the matrix material partly accounts for increased protein leakage from high-capacity, single-point attached recombinant protein A affinity matrices; the modifications in amino acid sequence introduced into such recombinant protein A as compared to full-length wild-type protein A contribute considerably to increased protein leakage, too.

3. Parallel Testing: Comparison with Miles Method (U.S. Pat. No. 4,983,722)

The Miles patent (U.S. Pat. No. 4,983,722) claims that DEAE Sepharose used as a second chromatography step in a binding mode with a salt gradient (0.025M to 0.25M NaCl) for elution can reduce the leached Protein A content in the eluate to less than 15 ng/mg antibody (range of protein A was 0.9 to 14 ng/mg of antibody).

TABLE 2
Comparison of Protein A residues in eluate samples
of 6A1 Antibody purified on single and multipoint
attached Protein A affinity matrices

| | | Protein A levels |
|---|---|---|
| Matrix | Sample | (ng/mg) |
| rProtein A Sepharose | Protein A eluate | 20.2 |
| (single point attached) | | |
| rmp Protein A Sepharose | Protein A eluate | 2.16 |
| (multi-point attached) | | |
| Native Protein A | Protein A eluate | <2.0 |
| Sepharose | | |
| (multipoint attached) | | |

The aim of these experiments was to confirm these results using MabSelect (new single point attached rProtein A matrix) with a lower pI antibody (pI 6.5-7.5), and to directly compare the non-binding Q-Sepharose method (using different equilibration/loading buffers) with the Miles patent method. The 6A1 antibody harvested from NSO cells and respective cell culturing methods for expression and harvest of antibody are described and referred to in more detail in experimental section 7 below.

### Method Applied:

The purification of 6A1 antibody (pI 6.5-7.5) included two chromatography steps consisting of MabSelect Protein A step followed by Q-Sepharose anion exchange chromatography (non-binding), or DEAE Sepharose chromatography (binding) step.

### MabSelect Protein A Chromatography:

| | |
|---|---|
| Column matrix | Mab Select recombinant Protein A (single |
| | point attached rPA) |
| Column dimensions | 1.6 cm internal diameter × 15 cm bed height |
| Column volume | 30 mL |
| Operational flow rate | 500 cm/hr (16.80 mL/min) |
| Clean | 6M guanidine HCL (2 column volumes) |
| Loading capacity | 35 mg/ml matrix |
| Equilibration | 50 mM glycine/glycinate pH 8.0/250 mM NaCL |
| | (8 column volumes) |
| Post load wash | 50 mM glycine/glycinate pH 8.0/250 mM NaCL |
| | (8 column volumes) |
| Elution buffer | 100 mM glycine pH 3.50 (6 column volumes) |
| Wash | 100 mM Citric acid pH 2.1 (2 column volumes) |

The culture supernatant containing 6A1 antibody was purified on a MabSelect column (30 ml), connected to an AKTA FPLC system. The conditions used were as described in the table above. The antibody was eluted using 0.1M glycine pH 3.5. Following elution the eluate pH was adjusted to pH 7.0, and then the eluate sample was divided into 5 aliquots; each aliquot was then diafiltered into a different buffer for anion exchange chromatography.

The first aliquot was diafiltered into 50 mMTris HCl pH8/75 mM NaCl for Q-Sepharose chromatography run 1. The second aliquot was diafiltered into 50 mMTris HCl pH8/100 mM NaCl for Q-Sepharose chromatography Run 2. The third aliquot was diafiltered into 20 mM sodium phosphate pH6.5/80 mM NaCl for Q-Sepharose chromatography Run 3. Aliquots four and five were buffer exchanged into 25 mMTris HCl pH 8.0/25 mM NaCl for evaluation of binding DEAE Sepharose method described in Miles patent. The difference between Runs 4 & 5 is that in Run 4 the main peak was collected as one fraction and diafiltered into standard phosphate buffered saline prior to analysis whereas in Run 5, the elution peak was fractionated and dialysed into a phosphate buffer prepared as described in the Miles patent.

The conditions for each of the five column runs are described below:

### O—Sepharose Chromatography: Run 1

| | |
|---|---|
| Column matrix | Q-Sepharose Fast Flow (Amersham Biosciences) |
| Column dimensions | 1.6 cm internal diameter × 8 cm bed height |
| Column volume | 16 mL |
| Column preparation | Packed in 0.1 M Sodium Hydroxide at 150 cm/hr |
| Operational flow rate | 100 cm/hr (3.35 mL/min) |
| Clean | 0.1M Sodium Hydroxide (2 column volumes) |
| Loading capacity | 15 mg/ml matrix |
| Equilibration | 50 mM TrisHCl pH 8.0/7 5 mM NaCl (8 column |
| | volumes) |
| Post load wash | 50 mM TrisHCl pH 8.0/75 mM NaCl (5 column |
| | volumes) |
| Strip buffer | 2 M Sodium Chloride (2 column volumes) |
| Wash | 0.1M Sodium Hydroxide (2 column volumes) |

### O—Sepharose Chromatography: Run 2

| | |
|---|---|
| Column matrix | Q-Sepharose Fast Flow (Amersham Biosciences) |
| Column dimensions | 1.6 cm internal diameter × 8 cm bed height |
| Column volume | 16 mL |
| Column preparation | Packed in 0.1 M Sodium Hydroxide at 150 cm/hr |
| Operational flow rate | 100 cm/hr (3.35 mL/min) |
| Clean | 0.1M Sodium Hydroxide (2 column volumes) |
| Loading capacity | 7.5 mg/ml matrix |
| Equilibration | 50 mM TrisHCl pH 8.0/100 mM NaCl (8 column |
| | volumes) |
| Post load wash | 50 mM TrisHCl pH 8.0/100 mM NaCl (5 column |
| | volumes) |
| Strip buffer | 2 M Sodium Chloride (2 column volumes) |
| Wash | 0.1M Sodium Hydroxide (2 column volumes) |

### O—Sepharose Chromatography: Run 3

| | |
|---|---|
| Column matrix | Q-Sepharose Fast Flow |
| Column dimensions | 1.6 cm internal diameter × 8 cm bed height |
| Column volume | 16 mL |
| Column preparation | Packed in 0.1 M Sodium Hydroxide at 150 cm/hr |
| Operational flow rate | 100 cm/hr (3.3 5 mL/min) |
| Clean | 0.1M Sodium Hydroxide (2 column volumes) |
| Loading capacity | 7.5 mg/ml matrix |
| Equilibration | 20 mM Sodium phosphate pH 6.5/80 mM NaCl |
| Post load wash | 20 mM Sodium phosphate pH 6.5/80 mM NaCl |
| | (5 column volumes) |
| Strip buffer | 2M Sodium Chloride (2 column volumes) |
| Wash | 0.1M Sodium Hydroxide (2 column volumes) |

### DEAE Sepharose: Run 4

| | |
|---|---|
| Column matrix | DEAE Sepharose (Amersham Biosciences) |
| Column dimensions | 1.6 cm internal diameter × 8 cm bed height |
| Column volume | 16 mL |
| Column preparation | Packed in equilibration buffer at 150 cm/hr |
| Operational flow rate | 100 cm/hr (3.35 mL/min) |
| Clean | 0.1M Sodium Hydroxide (2 column volumes) |
| Loading capacity | 7.5 mg/ml matrix |
| Equilibration | 25 mM TrisHCl pH 8.6/25 mM NaCl |
| | (8 column volumes) |
| Post load wash | 25 mM TrisHCl pH 8.6/25 mM NaCl |
| | (5 column volumes) |
| Elution buffer | 25 mM TrisHCl pH 8.6/25 mM NaCl To 25 mM |
| | TrisHCl pH 8.6/250 mM NaCl |
| | (10 column volumes) |
| Wash | 2M Sodium Chloride |
| | (2 column volumes) |

### DEAE Sepharose Binding Method: Run 5 (Miles Method)

| | |
|---|---|
| Column matrix | DEAE Sepharose |
| Column dimensions | 1.6 cm internal diameter × 8 cm bed height |
| Column volume | 16 mL |
| Column preparation | Packed in equilibration buffer at 150 cm/hr |
| Operational flow rate | 100 cm/hr (3.35 mL/min) |
| Clean | 0.1M Sodium Hydroxide (2 column volumes) |
| Loading capacity | 7.5 mg/ml matrix |
| Equilibration | 25 mM TrisHCl pH 8.6/25 mM NaCl |
| | (8 column volumes) |
| Post load wash | 25 mM TrisHCl pH 8.6/25 mM NaCl |
| | (5 column volumes) |
| Elution buffer | 25 mM TrisHCl pH 8.6/25 mM NaCl To |
| | 25 mM TrisHCl pH 8.6/250 mM NaCl |
| | (10 column volumes) |
| Wash | 2M Sodium Chloride |
| | (2 column volumes) |

The properties of the different buffers used in this study are shown in Table 3.

Eluate samples generated from the 5 ion exchange runs were assayed for Protein A levels in the rPA ELISA. The results are shown in Table 4.

TABLE 3
Buffers used in this study

| Equilibration | Run | Conductivity | | |
|---|---|---|---|---|
| Buffer | number | (ms/cm) | Resin | pH |
| 50 mM TrisHCl pH | 1 | 10.74 | Q-Sepharose | 8.00 |
| 8.0/75 mM NaCl | | | (non-binding) | |
| 50 mM TrisHCl pH | 2 | 12.85 | Q-Sepharose | 8.01 |
| 8.0/100 mM NaCl | | | (non-binding) | |
| 20 mM Sodium | 3 | 10.20 | Q-Sepharose | 6.50 |
| phosphate pH 6.5/ | | | (non-binding) | |
| 80 mM NaCl | | | | |
| 25 mM TrisHCl pH | 4/5 | 3.35 | DEAE- | 8.60 |
| 8.6/25 mM NaCl | | | Sepharose | |
| | | | (binding) | |
| 25 mM TrisHCl pH | 4/5 | 24.54 | DEAE- | 8.61 |
| 8.6/250 mM NaCl* | | | Sepharose | |
| | | | (binding) | |

*Gradient elution buffer

Fractions across the elution profile of DEAE-Sepharose Run 5 (Miles method) were collected and analysed in the rProtein A ELISA; the results are shown in Table 5.

TABLE 4
rProtein A ELISA Results:

| | rProtein A | Antibody | | Elution |
|---|---|---|---|---|
| | levels | concentration | % | Volumes |
| Sample ID | (ng/mg) | (mg/ml) | Recovery | (CV's)* |
| Q-Sepharose | <0.4 | 1.42 | 82 | 4.5 |
| eluate Run 1 | | | | |
| Q-Sepharose | 2.94 | 1.49 | 70 | 3.5 |
| eluate Run 2 | | | | |
| Q-Sepharose | 0.73 | 1.86 | 85 | 3.4 |
| eluate Run 3 | | | | |
| DEAE Sepharose | 1.72 | 2.16 | 75 | 2.5 |
| eluate pool Run 4 | | | | |
| (pool of all | | | | |
| fractions) | | | | |
| DEAE Sepharose | 1.55 | 1.83 | 73 | 3 |
| eluate pool (Miles | | | | |
| Method) Run 5 | | | | |
| (pool of fractions | | | | |
| 2 to 6) | | | | |

*Where CV's denotes column volumes

TABLE 5
Levels of rProtein A in Eluate fractions across
the elution peak obtained during binding-mode DEAE-Sepharose
separation (Miles Method); Run 5.

| Fraction | rProtein A levels | Absorbance |
|---|---|---|
| Number | (ng/mg) | (A₂₈₀) |
| 1 | 3.33 | 0.018 |
| 2 | 0.4 | 0.108* |
| 3 | 0.4 | 0.22* |
| 4 | 0.4 | 0.169* |
| 5 | 2.01 | 0.092 |
| 6 | 16.7# | 0.042 |
| 7 | 6.38 | 0.016 |

Miles's method, table 5: Whereas the main protein and hence antibody peak is in fractions 2-4 (start of numbering arbitrary; said fractions marked with a *) of the eluate, protein A retardeldy eluates in a sharply ascending peak (fraction marked with #); cutting of antibody recovery after fraction 4 at the very latest removes most of the aggregate, though at the expense of about 35% of the antibody found in the eluate above not being recoverable in view of complying with an admissible threshold for protein A contaminant of up to 2 ng/mg antibody.

In contrast, the non-binding method of Runs 1-3 allowed of excellent recovery of antibody in view of protein A contents criterium. Always, the non-binding methods yielded a sharp antibody protein peak as it is obtainable with the traditional binding methods, without any characteristic deformation of peak shape. It is to be noted that of course, the volume of the load does not suffice to have an antibody sample migrate and flow off from an exchanger column due to the much larger void volume. Hence the mobile phase feed that comes after the loading is denoted in the protocols above as ‘post loading wash’ for the present non-binding method, too. Along with the loading buffer front migrating through the column ahead, it generates the flow-through collected from the column in which the antibody product peak is encompassed, taking column void volume into account. Hence prior to collecting the product protein peak, always one column volume (the equilibration buffer) will come down which will never encompass product protein. The method of the present invention does not require an elution buffer, and it goes without saying that despite resemblance of terms, for the non-binding method of the present invention and as exemplified in Runs 1-3, such post-loading wash does still not allow of static binding of antibody or product protein, this in contrast to the post-loading buffer conditions according to Miles; in theory, for the method of the present invention the post-loading wash buffer could even be different from the loading buffer, as long as the afore mentioned non-binding condition requirement is preserved, but there would be no added benefit in doing so of course. Still then, all such buffers would give rise to the flow-through collected after passage through the column. Hence in Runs 1-3, the loading and post-loading wash buffers are the same for sake of simplicity. In Runs 1-3, the antibody peak was usually coming down in the flow-through method at about 1 to 2 column void volumes, typically at about 1.5 column volumes. But even under non-binding conditions that produced ‘elution’ of the product peak in the flow-through at about 2 to 3 column void volumes (data not shown), still no peak broadening or trailing was observable, indicating non-binding conditions were consistently operating. In the context of the non-binding method of the present invention and the experimental teaching of this paragraph, the indexed term ‘elution’ volume is used for this, as to oppose the term to a true binding-and-elute mode of operation according to Miles.—The highest antibody recovery (85%) for this antibody (6A1; pI 6.5-7.5) was obtained under non-binding conditions on Q-Sepharose using 20 mM sodium phosphate pH 6.5/80 mM NaCl buffer (corresponding to Run 3). Run 1 also showed good recovery (82%) however, the ‘elution’ volume for this run was somewhat higher whilst no substantial broadening of the antibody protein peak could be observed though; glycoform distribution was not analyzed. Increasing the NaCl concentration (Run 2 vs. 1) resulted in lower rProtein A clearance, hence the buffer systems used in Runs 3 and 1 were more appropriate for this antibody. It has been our previous observation that the buffer system used in Run 1 is more appropriate for high pI antibodies and that one used in Run 3 tends to be more useful for neutral or slightly acidic antibodies. Given the data from Run 1, one can expect to use this non-binding method at even much higher capacities (>30 mg/ml). The non-binding process allows more easily of large scale production as compared to the Miles method as higher capacity etc. can be applied, apart from circumventing one major drawback of Miles' method, namely the need of paying meticulous care to fractionation of eluate for the purpose of avoiding protein A peak fractions alone; the latter would become even more difficult, if not impossible, once multiple parameters (aggregate plus proteinA contaminants thresholds) would need to be complied with in combination at the same time:

In the case of Run 5 (Miles method), fractionation of rProtein A was observed across the main elution peak as shown in table 5. Careful pooling of fractions is therefore required to ensure good clearance of rProtein A. This had an impact on recovery (70%) and even in this case did not give as good clearance as obtained with the non-binding method. For the Miles method therefore it is more difficult to achieve good clearance and high recovery for cell lines/antibodies in cases where very high leakage is observed (such as that commonly obtained with single point attached matrices).

The data from Run 5 is representative of the results obtained by and the conditions described in the Miles patent.

An overview of method comparisons and the data obtained is shown in Table 6, below.

TABLE 6.1
Summary of rProtein A Levels at Different Stages of Antibody Purification*

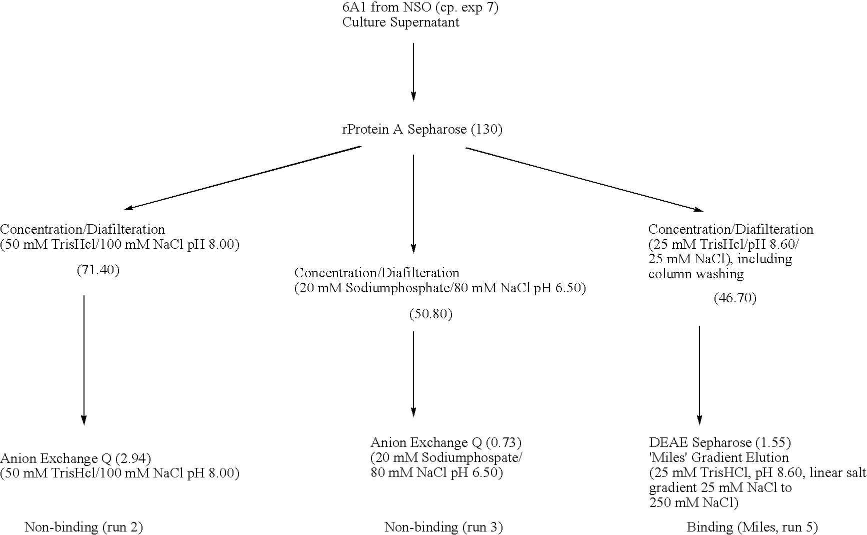

Note:
Levels of rProtein A are shown in brackets [ng/mg]; note that not all NSO clonal cell lines’ supernatants give similar contamination levels of protein A.
All examples carried out with 7.5 mg/ml loading of anion exchangers

Similar to Run 2 on the far left in table 6.1, Run 1 was conducted in a non-binding mode but with 15 mg/ml loading capacity and further decreased ionic strength (table 6.2), resulting in excellent derichment of contaminating protein A:

TABLE 6.2
(Run 1)

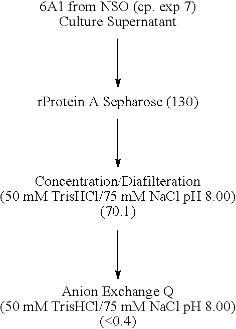

It was found that this excellent result using Anion Exchange Q-Sepharose was fully reproducible, also using different e.g. high pI antibody.

### 6. Purification Using Ceramic Q-HyperD® F as an Anion Exchanger

Essentially, non-binding anion exchange chromatography with eluate from a Mab-Select protein A chromatography was carried out as described in comparative experiment 3 above. Q-HyperD® F (Biosepra-Brand of chromatographic supports) was purchased from Ciphergen Biosystems Ltd., Guildford, UK. The processing of a pI 8-9 antibody expressed from NSO cells by Mab-Select Protein A affinity chromatography was conducted essentially as described in example 5. Further essentially as described in example 5 (for Runs 1-3), Q anion exchange chromatography in flow-through mode was then applied to the Protein A-affinity column eluate except that Q-Sepharose, except for a comparative run, was replaced by Q-Hyper DF (Biosepra®) under varying conditions of buffer salt, buffer pH and conductivity. The respective conditions are outlined in the scheme according to Table 7; applying a very low conductivity of less than 2 mS/cm, namely at about 1.26 mS/cm, proved superior with regard to deriching contaminating protein A to the utmost extend possible and achieving results equal to those obtainable with Sepharose Q material. Tailing of aggregates (data not shown) in the flow-through fractions was analytically observed as well, its extend also being dependent on the buffer solution applied. Depending on the primary objective and the type of ion exchange material, single best or compromise conditions for buffer definition must be defined for a given separation task. However, for large scale industrial manufacture, the ceramic HyperD material offers advantages in view of life time, robustness and compressibility (processing time, flow rate). Hence conductivity is a very important parameter to be tested and optimized for different column materials. It is also to be noted that the conductivity quite severely affects contaminant levels of DNA which is a polyanion. By suitably fine-tuning the buffer conditions, both contaminant DNA and protein A levels can be jointly and concomitantly reduced to the utmost degree.

TABLE 7
Q-Hyper D ®- Evaluation

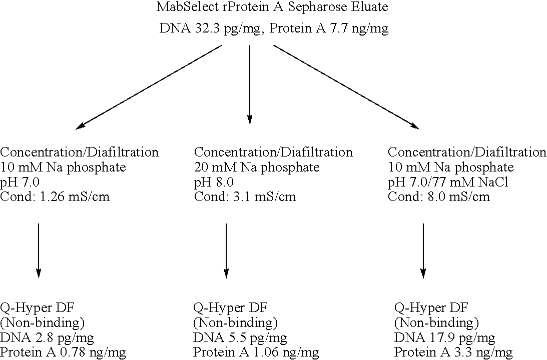

For comparison: Using the same antibody, Q-Sepharose gave 0.4 ng protein A/mg antibody, contaminant DNA level was determined with 10.9 pg/mg antibody; however, this result on Q was achieved with a quite different buffer (20 mM Tris HCl/50 mM NaCl pH 8.00, amounting to a conductivity measured of 6.1 mS/cm).

7. Use of Ion Exchange Chromatography in a Non-Binding Mode for Concomitant Aggregate and Protein A Reduction after Protein A Chromatography

The aim of these experiments was to evaluate aggregate-monomer separations (using cB72.3 IgG antibody having pI of pH 6.5-7.5 as harvested from clonal cell line NS0-6A1-Neo, a cell line carrying a glutamine synthetase (GS) and a neomycin selection marker and constitutively expressing antibody) across ion exchange chromatography operated in a non-binding mode. The matrix selected for evaluation was Q-Sepharose anion exchange (Amersham Biosciences) run under two different buffer conditions.

Culturing NS0-GS cells and harvesting B72.3 antibody has been described elsewhere in detail (cp. WO 03/027300 and WO 03/064630 of the same applicant). The producer cell line NS0-6A1 has been deposited under the code ‘6A1-Neo’ on Aug. 30, 2002 under the treaty of Budapest under accession number 02083031 at the European Collection of Cell Cultures (ECACC), Centre for Applied Microbiology and Research, Porton Down, Salisbury/Wiltshire SP4 0JG, United Kingdom on behalf of Andy Racher, Lonza Biologicals, 224 Bath Road, Slough, Berkshire, SL1 4DY, United Kingdom; the address given is the company address of Lonza Biologics plc., United Kingdom and the commission has been carried out on commission of and with all rights vested in Lonza Biologics plc. To the extend Mr. Andy Racher, whose current private address is 5 Kingfisher Close, Aldermaston, Reading/Berkshire RG7 4UY, United Kingdom, may be occasionally deemed to be the lawful depositor, it is declared that with regard to such legal interpretation of the deposit documents, Mr. Racher has unreservedly and irrevocably authorised the present applicant, Lonza Biologics plc., to refer to the deposited material in the application and to make it available to the public and has assigned all title in the deposit to the present applicant.

The gene structure of mouse-human chimeric antibody cB72.3 is described in Whittle et al., Protein Eng. 1987, 6: 499-505 and Colcher et al., Cancer Research 49, 1738-1745, (1989). The antibody is also expressed from NS0-6A1-Neo cell line. The purification process for NS0 6A1 antibody (cB72.3) includes two chromatography steps consisting of rmp Protein A Sepharose followed by non-binding Q-Sepharose anion exchange chromatography.

### rmp Protein A Sepharose Chromatography

| | |
|---|---|
| Column matrix | rmp Protein A Sepharose (Amersham Biosciences) |
| Column Dimensions | 1.8 cm internal diameters × 15 cm bed height |
| Column Volume | 30.1 ml |
| Operating Flow | 150 cm/hr |
| Rate | |
| Clean | 6M Guanidine HCL (2 column volumes) |
| Loading Capacity | 35 mg/ml matrix |
| Equilibration | 50 mM Sodium Phosphate pH 7.0/250 mM NaCl |
| | (8 Column Volumes) |
| Post Load Wash | 50 mM Sodium Phosphate pH 7.0/250 mM NaCl |
| | (8 Column Volumes) |
| Elution Buffer | 0.1M Glycine/O.1M NaCl pH 3.0 (6 column |
| | volumes) |
| Strip | 0.1M Citric Acid pH 2.1 (2 column volumes) |

Cell culture supernatant containing 6A1 antibody was purified on an rmp Protein A column (30 ml), connected to an ATKA FPLC system. The conditions used were as described in the table above. The antibody was eluted using 0.1M Glycine/0.1M NaCl pH3.0. Following elution the eluate was pH adjusted to pH 3.7, held for 60 minutes, and then neutralised to pH 6.5. It was necessary to perform two cycles. The eluate from the first cycle was concentrated to 25 mg/ml, buffer exchanged into 20 mM Na Phosphate/80 mM NaCl pH 6.5 and loaded onto a Q-Sepharose column under ‘Run 1’-elution conditions shown below. The eluate from the second cycle was concentrated to 25 mg/ml, and buffer exchanged into 20 mM Tris HCL/75 mM NaCl pH8.0 and applied to a Q-Sepharose column as described for Run 2 below.

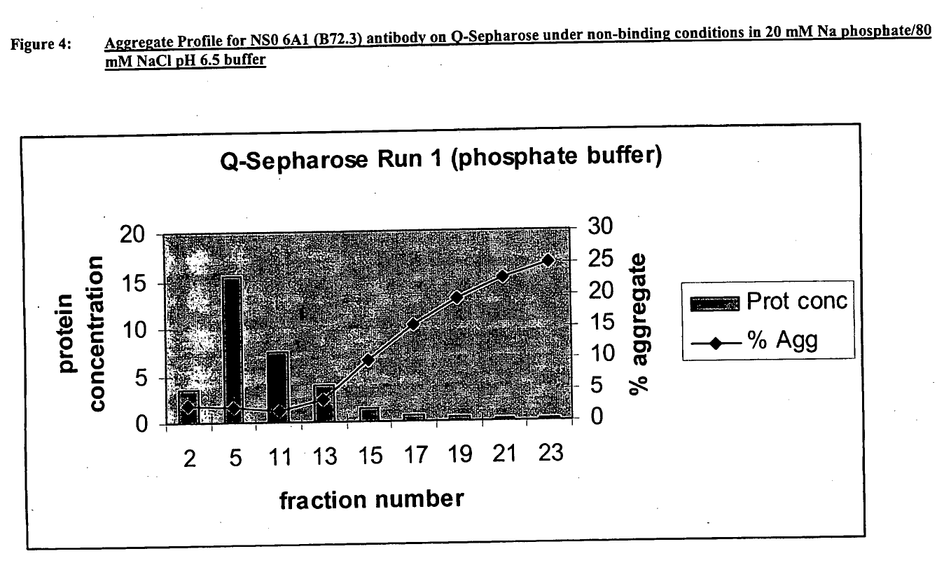

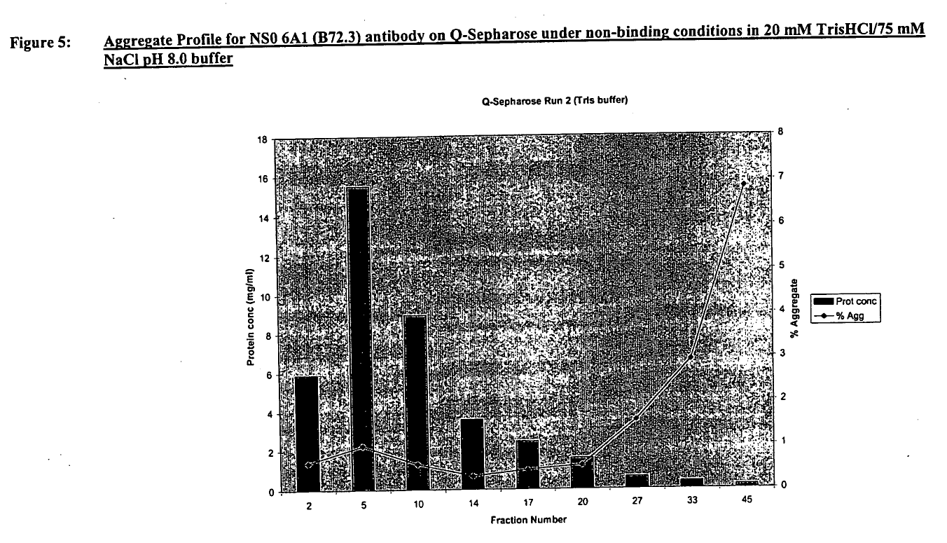

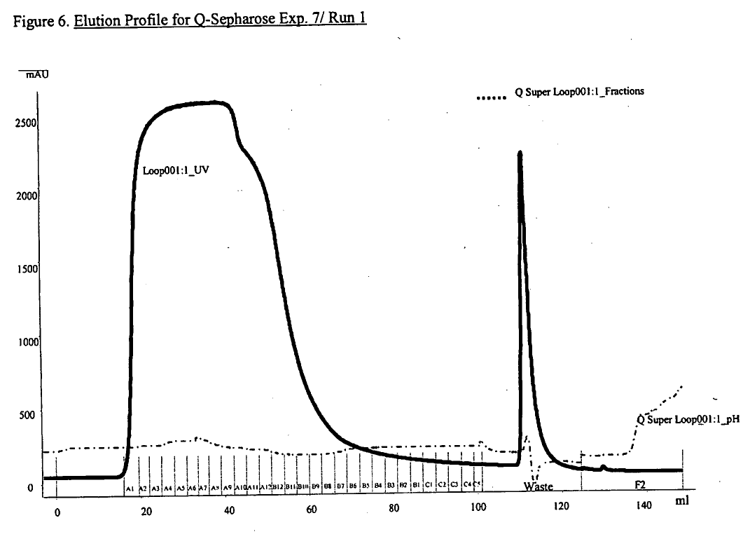

Fractions were collected across the unbound fraction (Followthrough) and were analysed by gel permeation-HPLC. The recoveries and elution volumes are presented in Table 1. The GP-HPLC results are presented in Table 2. The aggregate profiles are shown in FIGS. 4 & 5 and the elution profiles are shown in FIGS. 6 & 7.

### O-Sepharose Chromatography Run 1:

| | |
|---|---|
| Column matrix | Q-Sepharose FF (Amersham Biosciences) |
| Column Dimensions | 1.0 cm internal diameter × 15 cm bed height |
| Column Volume | 12 ml |
| Operating Flow | 100 cm/hr |
| Rate | |
| Clean | 0.1M Sodium Hydroxide (2 column volumes) |
| Loading Capacity | 50 mg/ml matrix |
| Equilibration | 20 mM Na Phosphate/80 mM NaCl ph 6.5 |
| Post Load Wash | 20 mM Na Phosphate/80 mM NaCl ph 6.5 |
| Strip | 20 mM Na Phosphate/2M NaCl ph 6.5 |
| | (2 column volumes) |

The UV-monitored chromatogram is shown in FIG. 6.

### Q-Sepharose Chromatography Run 2:

| | |
|---|---|
| Column matrix | Q-Sepharose FF (Amersham Biosciences) |
| Column Dimensions | 1.0 cm internal diameter × 15 cm bed height |
| Column Volume | 12 ml |
| Operating Flow | 100 cm/hr |
| Rate | |
| Clean | 0.1M Sodium Hydroxide (2 column volumes) |
| Loading Capacity | 50 mg/ml matrix |
| Equilibration | 20 mM Tris HCL/75 mM NaCl pH 8.0 |
| Post Load Wash | 20 mM Tris HCL/75 mM NaCl pH 8.0 |
| Strip | 20 mM Tris HCL/2M NaCl pH 8.0 (2 column |
| | volumes) |

| | | Elution Vol |
|---|---|---|
| Run Number | Recovery (%) | (cv) |
| Q-Sepharose | 73 | 5.8 |
| Run 1 Eluate | | |
| Q-Sepharose | 77 | 11 |
| Run 2 Eluate | | |

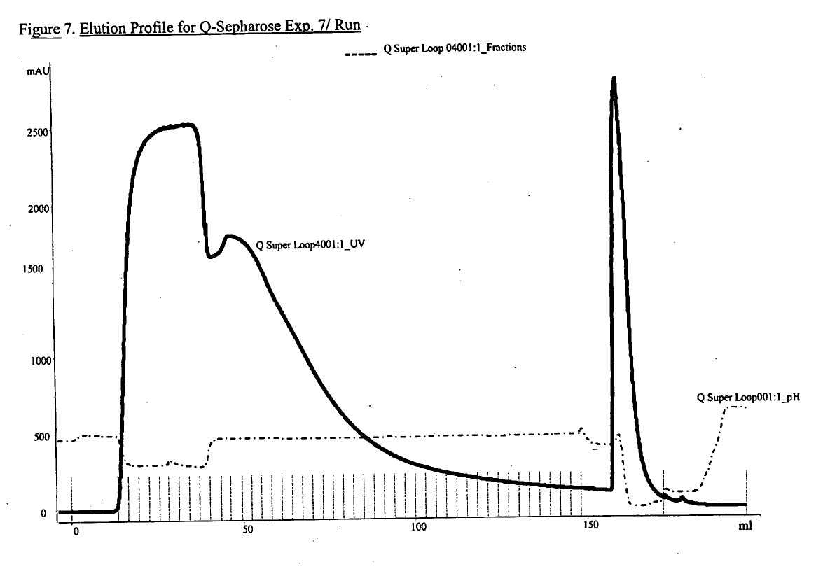

The UV-monitored chromatogram (OD at 260 nm) is shown in FIG. 7.

For gel permeation/size exclusion chromatography, redundant triple detection (RALS, Viscometer and Refractive Index) was applied for detecting the protein fractions coming of the gel column: The light scattering detector provides a direct measurement of the molecular weight and eliminates the need for a column calibration. The viscometer allows differences in structure to be seen directly. It also allows the molecular size to be determined across the entire distribution. One additional advantage of triple detection is that the instrument parameters can be determined by using a single narrow and a single broad standard. Triple detection determines the “absolute” molecular weight, intrinsic viscosity and molecular size in a single measurement. It provides information on branching, conformation, structure and aggregation of the polymer sample.

### Chromatographic Conditions:

| | |
|---|---|
| Solvent: | 0.2M Sodium Phosphate buffer, pH = 7.0 |
| Flow rate: | 0.7 ml/min |
| Injection volume: | 100 μl |
| Column/Detector temperature: | 29° C. |
| Columns: | Superdex 200HR |
| Detector: | |

The triple detection chromatograms of the sample showed excellent signal to noise on the detectors. The reproducibility of the monomer peak is very good. For all samples Mw was around 140 k-147 k Dalton, intrinsic viscosity IV˜0.065-0.079 dl/g, hydrodynamic radius Rh˜5.3-5.5 nm and weight fraction 80-99%. The second peak has molecular weight around 300 k, IV˜0.08 dl/g and Rh˜7 nm, which would agree with results of Dimer.

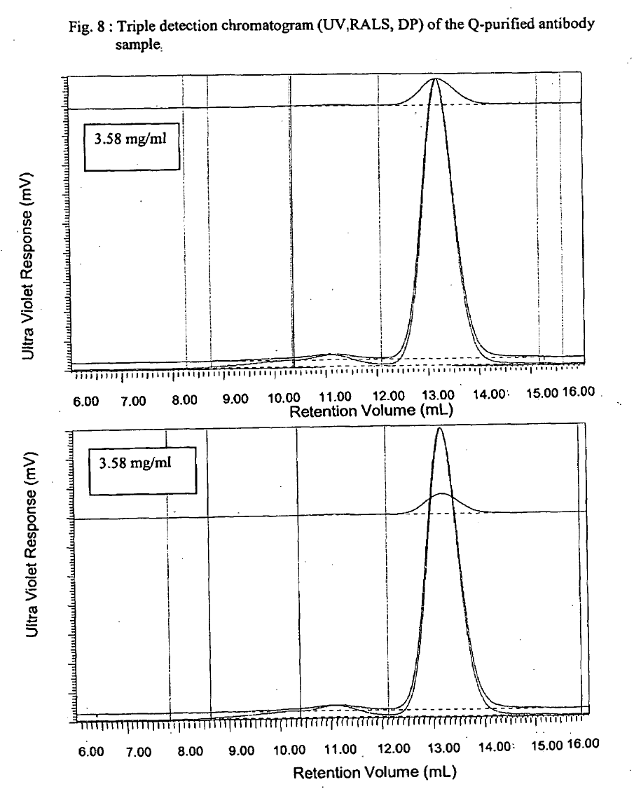

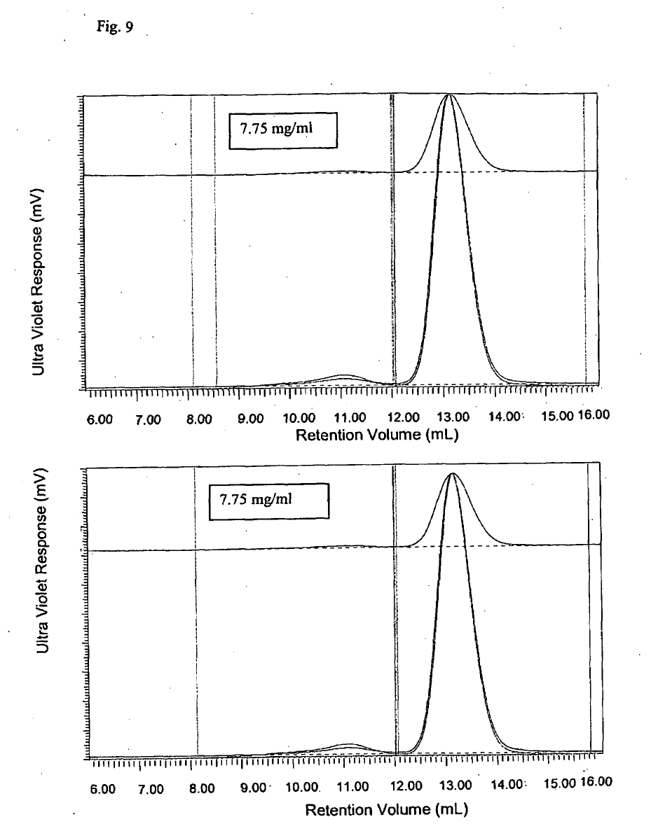

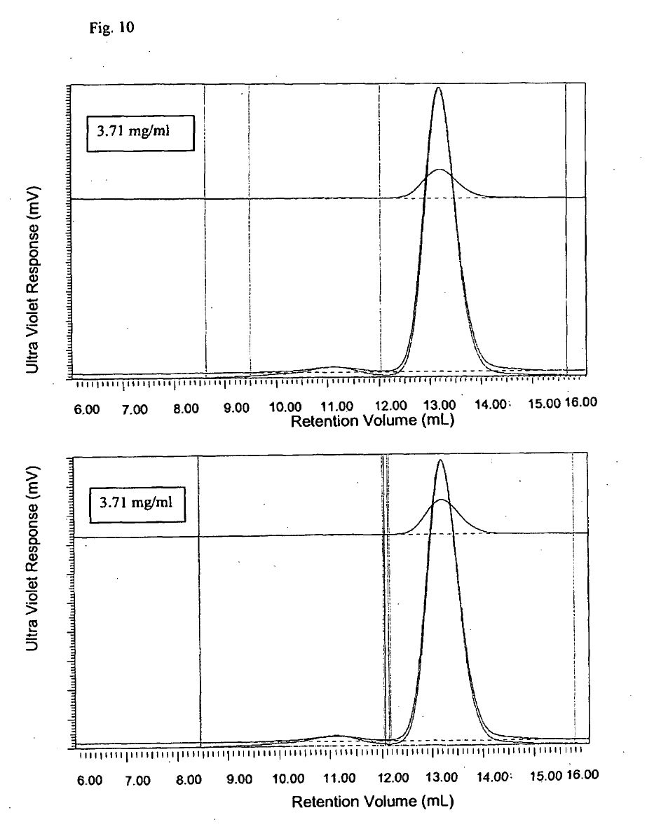

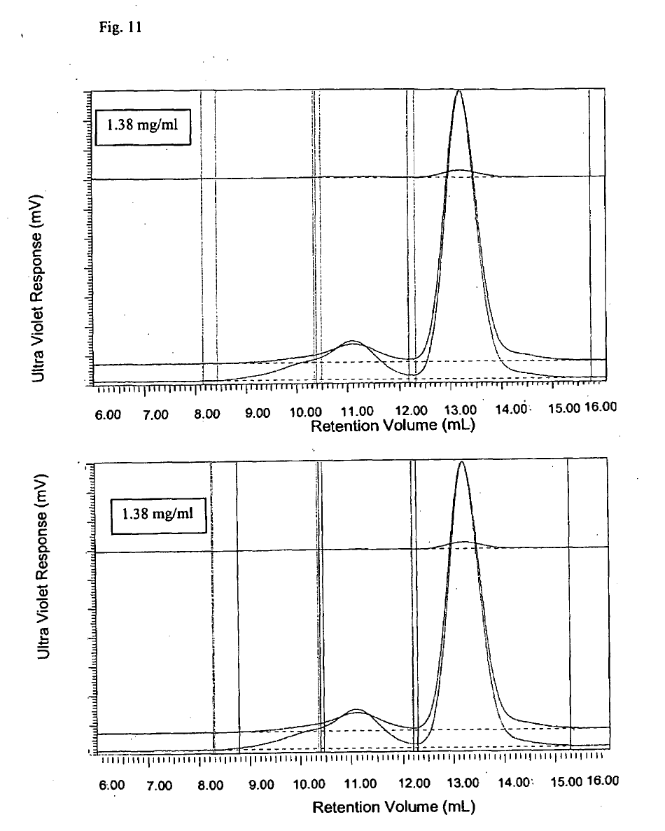

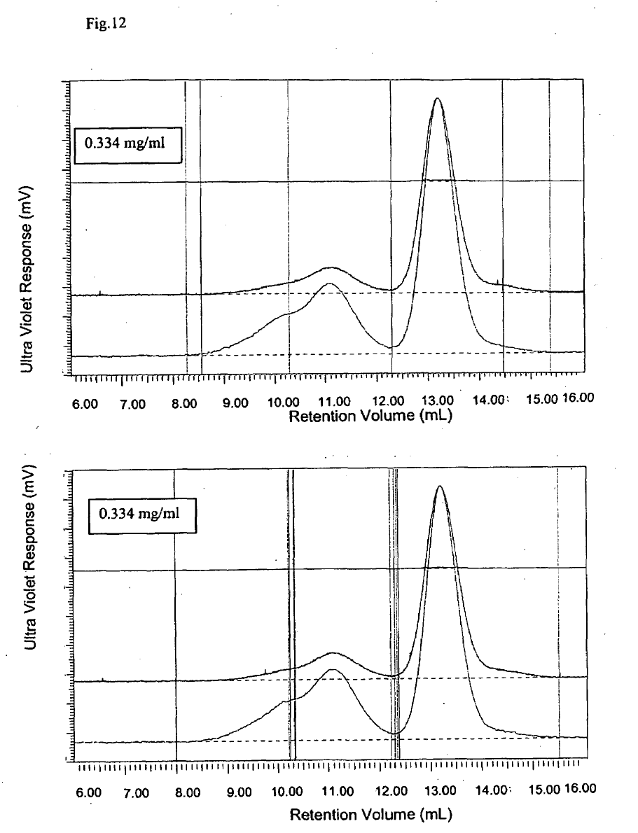

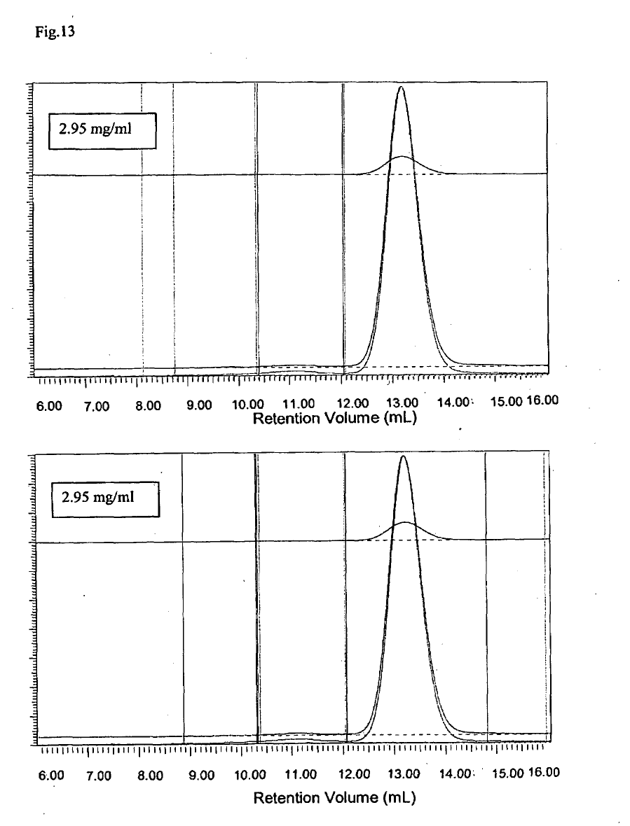

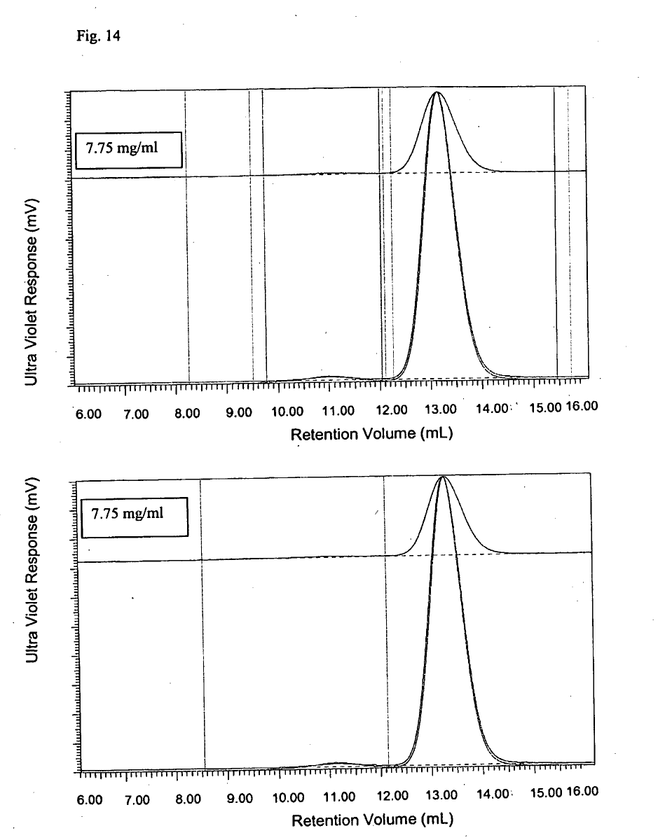

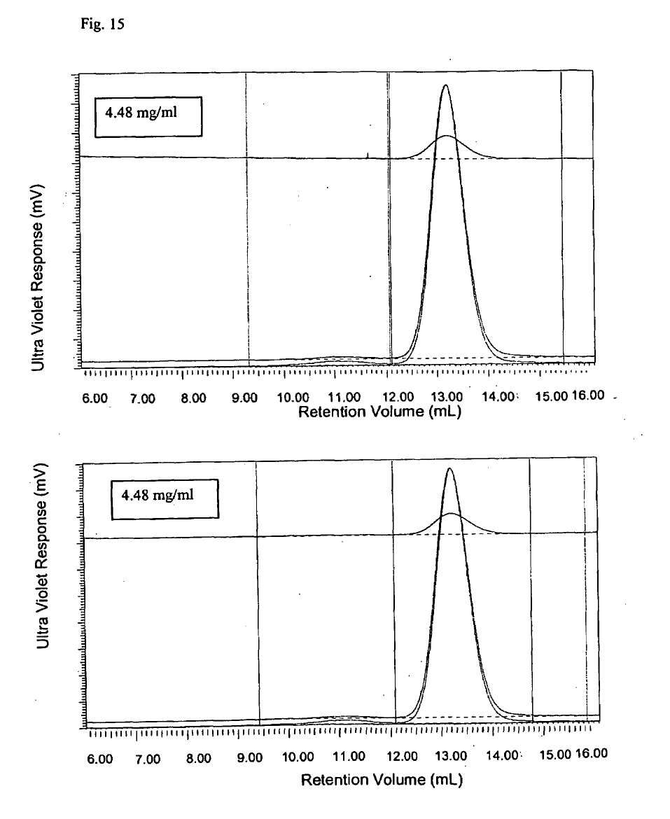

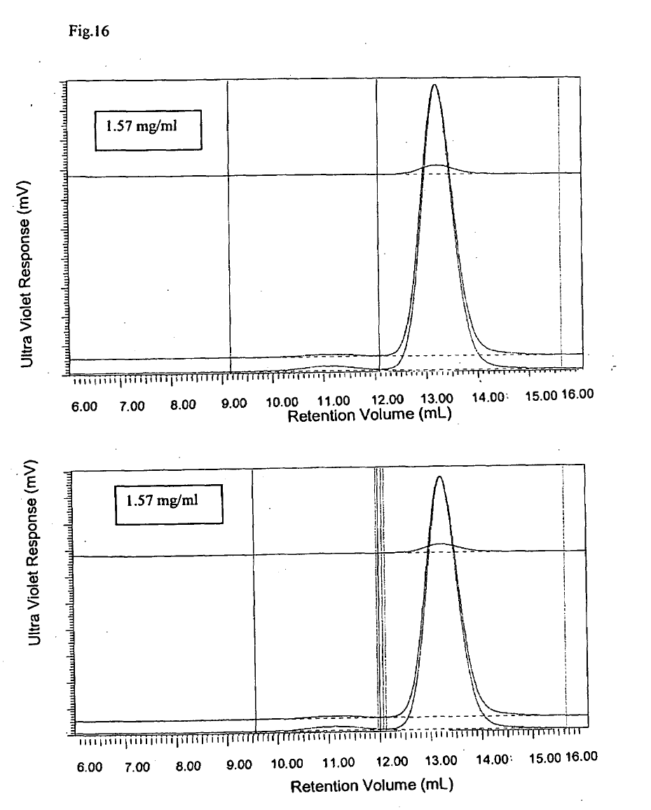

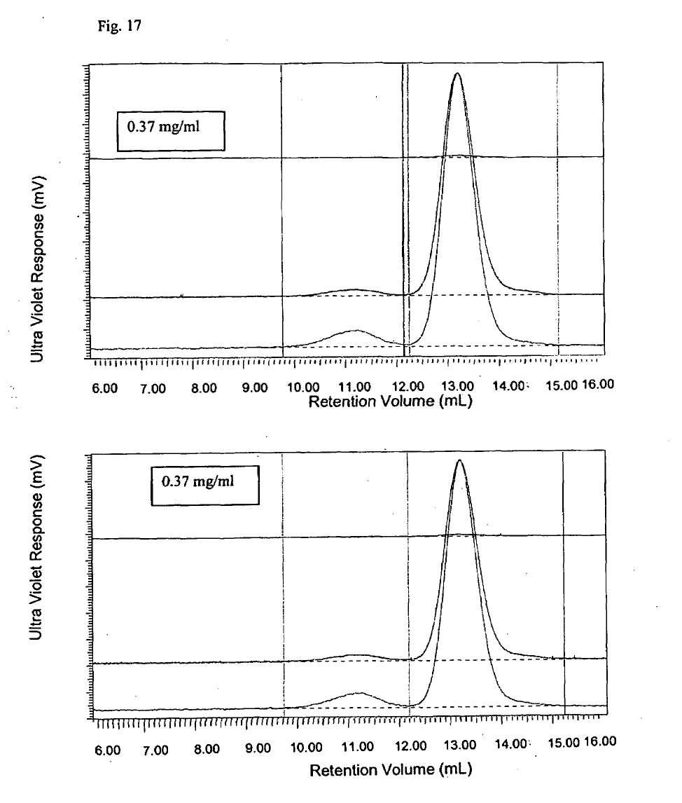

FIG. 8-17 show duplicate GP chromatography runs with triple detection for selected fractions from Table 2 (cp. concentration data. for cross-referencing).

| | | | |
|---|---|---|---|
| FIG. 8/Fraction | Q-FT-01-F2 | conc. = 3.58 mg/ml | |
| 1.2 | | | |
| FIG. 9/F. 1.5 | Q-FT-01-F5 | conc. = 15.5 mg/ml | *7.75 mg/ml |
| FIG. 10/F. 1.11 | Q-FT-01-F11 | conc. = 7.41 mg/ml | *3.71 mg/ml |
| FIG. 11/F. 1.15 | Q-FT-01-F15 | conc. = 1.38 mg/ml | |
| FIG. 12/F. 1.19 | Q-FT-01-F19 | conc. = 0.334 mg/ml | |
| FIG. 13/F. 2.2 | Q-FT-02-F2 | conc. = 5.9 mg/ml | *2.95 mg/ml |
| FIG. 14/F. 2.5 | Q-FT-02-F5 | conc. = 15.5 mg/ml | *7.75 mg/ml |
| FIG. 15/F. 2.10 | Q-FT-02-F10 | conc. = 8.95 mg/ml | *4.48 mg/ml |
| FIG. 16/F. 2.20 | Q-FT-02-F20 | conc. = l. 57 mg/ml | |
| FIG. 17/F. 2.33 | Q-FT-02-F33 | conc. = 0.37 mg/ml | |

*½ dilutions with phosphate buffer.

TABLE 2
Results of GP-HPLC Analysis: Aggregate Profiles

| | Protein Concentration | |
|---|---|---|
| Fraction Number | mg/ml | % Aggregate |
| Run 1 Load | 18.37 | 3.8 |
| 2 | 3.58 | 2.6 |
| 5 | 15.5 | 2.4 |
| 11 | 7.41 | 1.8 |
| 13 | 3.92 | 3.6 |
| 15 | 1.38 | 9.7 |
| 17 | 0.62 | 15.4 |
| 19 | 0.33 | 19.3 |
| 21 | 0.23 | 22.7 |
| 23 | 0.17 | 25.0 |
| Pooled Eluate | 6.36 | 2.9 |
| Run 2 Load | 20.7 | 3.9 |
| 2 | 5.9 | 0.6 |
| 5 | 15.5 | 0.99 |
| 10 | 8.95 | 0.6 |
| 14 | 3.59 | 0.3 |
| 17 | 2.44 | 0.4 |
| 20 | 1.57 | 0.5 |
| 27 | 0.61 | 1.6 |
| 33 | 0.37 | 2.9 |
| 45 | 0.19 | 6.8 |
| Pooled eluate | 3.4 | 0.9 |

In both Q-Sepharose runs buffered in 20 mM Na Phosphate/80 mM NaCl pH6.5 and 20 mM Tris HCL/75 mM NaCl pH8.0 relative high amounts of monomer were present in the early fractions with the majority of the aggregate eluting in the tail fractions. Run 1 (buffered in 20 mM Na Phosphate/80 mM NaCl pH6.5) contained higher levels of aggregate in the tail fractions in comparison to Run 2 buffered in 20 mM Tris HCL/75 mM NaCl pH8.0. Suitable aggregate-free fractions of the protein peak, avoiding peak fractions were pooled. The pooled antibody from said aggregate-free fractions was shown to be >99.1% monomeric by means of size exclusion HPLC. The level of contaminant protein A in the pooled monomer fractions is determined with Concomitantly, the level of contaminant protein A in the selected and pooled fractions is determined to be <<1.5 ng/mg antibody.

1. Method of purifying an antibody, preferably an IgG antibody, comprising the steps of:
1. Purifying an antibody by means of protein A affinity chromatography wherein the protein A is a native protein A or a functional derivative thereof,
2. loading the thus purified antibody comprising antibody aggregate and protein A or protein A derivative onto an ion exchange material under conditions which allow of binding of the contaminating protein A or its functional derivative to the ion exchanger material and which conditions further allow of resolution in the flow-through of antibody aggregates from antibody monomer which monomer is not complexed with protein A or protein A derivative by means of fractionation of the flow-through, and further
3. fractionating the flow-through and harvesting from the flow-through of the ion exchanger at least one antibody monomer fraction having both reduced contents of protein A or protein A derivative and further reduced contents of antibody aggregate as compared to the composition of antibody as loaded onto the ion exchange material before.

2. Method according to claim 1, characterized in that the protein A is a recombinant protein A that is engineered such as to allow of single-point attachment to a column material.

3. Method according to claim 2, characterized in that the recombinant protein A comprises a cysteine in its amino acid sequence.

4. Method according to claim 3, characterized in that the cysteine is comprised in a segment that consists of the last 30 Amino acids of the C-terminus of the amino acid sequence of the recombinant protein A.

5. Method according to claim 3, characterized in that the recombinant protein A is attached by at least 50% via a thioether sulphur bond to the chromatographic support material of the protein A affinity chromatography.

6. Method according to claim 3, characterized in that the protein A or its functional derivative is reduced to a concentration of <1 ng/mg IgG in the flow-through of the ion-exchanger.

7. Method according to claim 3, characterized in that the monomericity of the antibody harvested is at least 99% and is achieved by fractionating the antibody peak of the flow-through into at least two fractions and wasting the tail fraction.

8. Method according to claim 3, characterized in that the antibody is a monoclonal antibody, preferably an IgG antibody wherein the IgG antibody may be chimeric or CDR-grafted IgG antibody.

9. Method according to claim 3, characterized in that the antibody is harvested from a cell culture prior to purifying the antibody by means of protein A affinity chromatography.

10. Method according to claim 3, characterized in that the antibody is harvested from a mammalian cell culture.

11. Method according to claim 3, characterized in that the antibody that is to be purified by means of protein A affinity chromatography is not treated as to inactivate proteases, preferably is not in admixture with at least one protease inhibitor.

12. Method according to claim 3, characterized in that the protease inhibitor is selected from the group consisting of PMSF, a proteinase inhibiting peptide, e-caproic acid, and a reducing sulfhydryl compound.

13. Method of purifying a product protein, comprising the steps of:
1. loading a solution comprising product protein which product protein comprises monomeric and aggregated forms of said protein onto an ion exchange material under conditions which allow of resolution in the flow-through of said product protein aggregates from said product protein monomer which monomer preferably is not further complexed with a second protein ligand, by means of fractionation of the flow-through and further
2. fractionating the flow-through and harvesting from the flow-through of the ion exchanger at least one product protein monomer fraction having reduced contents of product protein aggregate as compared to the composition of product protein loaded onto the ion exchange material for purification.

14. Method according to claim 13, characterized in that fractionation is achieved by fractionating or splitting the product protein peak of the flow-through into at least two fractions and wasting the tail fraction and that, preferably, the monomericity of the antibody harvested is at least 99%.

15. Method according to claim 12, characterized in that at least one second fraction having a lower degree of monomericity of product protein than the first one is discarded based on the assessment of monomericity.

16. Method according to claim 15, characterized in that at least one buffer is used for loading and rinsing the ion exchanger which at least one buffer coming off the ion exchanger is constituting the flow-through comprising the product protein peak.

17. Method according to claim 16, characterized in that the pH of said buffer is set at a pH which is the pi or average pi of the product protein monomer sought to be purified in the range of ±0.5 pH units around said pi.

18. Method according to claim 16, characterized in that the pH of said buffer is set at a pH different from the pI or average pI of the product protein monomer sought to be purified and which pH further vests the product protein monomer with a surface charge which charge leads to ionic attraction in between product protein monomer and the charged groups of the ion exchange material when exposed to or submerged in said buffer.

19. Method according to claim 18, characterized in that in case of a cation exchanger, the pH of the buffer is set at a value below the average pi of the product protein monomer sought to be purified, preferably set at a value of from 0.5 to 3 pH units below said average pi.

20. Method according to claim 18, characterized in that in case of an anion exchanger, the pH of the buffer is set at a value above the average pi of the product protein monomer sought to be purified, preferably set at a value of from 0.5 to 3 pH units above said average pi.

21. Method according to claim 13, characterized in that said conditions are non-binding conditions as regards binding of the product protein monomer to the ion exchange material such as that consequently more than 70% (w/w), more preferably more than 80% (w/w) of the product protein loaded onto the ion exchange material can be recovered in the flow-through from the ion exchange material.

22. Method according to claim 13, characterized in that at least one second fraction having a lower degree of monomericity of product protein than the first one is discarded based on the assessment of monomericity.
